(19) United States
(12) Patent Application Publication
(10) Pub. No.: US 2011/0189303 A1
(43) Pub. Date: Aug. 4, 2011

(54) METHODS FOR ENHANCING THE QUALITY OF LIFE OF A SENIOR ANIMAL

(75) Inventors: Ryan Michael Yamka, Topeka, KS (US); Nolan Zebulon Frantz, Topeka, KS (US); Xiangming Gao, Topeka, KS (US); Kim Friesen, Carthage, IN (US)

(73) Assignee: Hill's Pet Nutrition, Inc., Topeka, KS (US)

(21) Appl. No.: 13/054,752

(22) PCT Filed: Jul. 20, 2009
(86) PCT No.: PCT/US2009/051155

§ 371 Date: Apr. 8, 2011

Related U.S. Application Data
(60) Provisional application No. 61/082,183, filed on Jul. 18, 2008.

(51) Int. Cl. A61K 33/42; A61K 33/32; A61P 39/00

(52) U.S. Cl. 424/601; 424/639

(57) ABSTRACT

The present invention relates to methods for enhancing the quality of life of a senior or super senior animal by feeding the animal a composition comprising at least one omega-3 polyunsaturated fatty acid and various combinations of amino acids, minerals, and antioxidants in amounts effective to enhance alertness, improve vitality, protect cartilage, maintain muscle mass, enhance digestibility, and improve skin and pelage quality. Beneficial changes in expression of genes associated with several biological pathways may be induced in an animal by feeding it said composition and are consistent with an enhancement in the quality of life of said animal.

### FIELD OF THE INVENTION

The present invention relates generally to methods for modulating biological functions associated with the aging process of an animal and particularly to using food compositions containing omega-3 polyunsaturated fatty acids for modulating biological functions associated with the aging process of a senior or super senior animal.

### BACKGROUND OF THE INVENTION

Companion animals such as dogs and cats frequently require differing diets depending on their life stage (age), size, body composition, and breed. Both dog and cat nutrient requirements can be separated into three different life-stages, based on age: growing dogs (or cats), adult dogs (or cats), and senior dogs (or cats). The latter category, senior dogs (or cats), can be further separated into two stages, which include senior (or mature adult) and super senior (or geriatric). Dogs are further separated into different categories for regular breed dogs versus large-breed dogs.

Essential fatty acids, consisting of omega-3 and omega-6 polyunsaturated fatty acids, are critical nutrients for the health of an animal. These nutrients, however, either cannot be made by animals or cannot be made in sufficient amounts to elicit benefits and therefore must be consumed in an animal's diet. See, e.g., Hornstra, G., et al., “Essential fatty acids in pregnancy and early human development”, Eur. J. Obs. & Gyn. and Reprod. Biology, 61:57-62 (1995). It has previously been postulated that Docosahexaenoic Acid (“DHA”), an omega-3 polyunsaturated fatty acid, is effective in increasing the maze-learning ability and brain functions in aged mice. See, Lim, S.-Y., “Intakes of dietary docosahexaenoic acid ethyl ester and egg phosphatidylcholine improve maze-learning ability in young and old mice”, J. Nutr., 130:1629-1632 (2000).

Rogers discusses the theory of the potential use of antioxidants to slow the deterioration of cognitive function, particularly in the elderly. See Rogers, P., “A healthy body, a healthy mind: long-term impact of diet on mood and cognitive function”, Proceedings of the Nutrition Society, 60:135-143 (2001).

Despite the studies and developments relating to improving cognitive abilities, there continues to be a need for methods for enhancing the quality of life of senior animals, as measured by, e.g., enhanced alertness, improved vitality, cartilage protection, maintenance of muscle mass, enhanced digestibility, and improved skin and pelage quality in senior and super senior animals.

As previously reported, the super senior pet food composition described herein may be administered to achieve this result. Additionally, we now report herein our surprising discovery that the enhanced quality of life of senior and super senior animals achieved by the administration of the pet food compositions disclosed herein is reflected at the genomic level. Specifically, as described in detail in the Examples below, gene chip data indicate that the expression of genes that encode proteins associated with several biological pathways such as blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport are modified, i.e., in general, the majority are beneficially altered through administration to the animal of the super senior pet food compositions described herein.

### SUMMARY OF THE INVENTION

The invention encompasses methods for improving or enhancing the quality of life of senior and super senior animals by feeding the animal a composition comprising at least about 9% by weight protein, at least about 5% by weight fat, and at least about 0.05% by weight of at least one omega-3 polyunsaturated fatty acid.

In one embodiment, the invention encompasses compositions effective to enhance an animal's quality of life, wherein enhanced quality of life is evidenced by improvement in one or more characteristics chosen from alertness, vitality, cartilage protection, muscle mass maintenance, digestibility, and skin and pelage quality.

In another embodiment, the invention encompasses compositions comprising at least one omega-3 polyunsaturated fatty acid chosen from docosahexaenoic acid (“DHA”) and eicosapentaenoic acid (“EPA”). In an additional embodiment, the method comprises feeding the animal a composition further comprising at least one antioxidant and at least one nutrient chosen from choline, manganese, methionine, cysteine, L-carnitine, lysine, and mixtures thereof.

In one embodiment, the invention encompasses compositions effective to improve or enhance the animal's quality of life, wherein enhanced quality of life is evidenced by improvement in one or more biological pathways chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport.

In another embodiment, the invention encompasses compositions effective to enhance the animal's quality of life, wherein enhanced quality of life is evidenced by a beneficial change in expression of one or more genes which encode proteins associated with or related to biological pathways chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport.

In yet another embodiment, the invention encompasses methods to treat an animal suffering from a disorder or disease associated with or related to a biological pathway chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport comprising administering to said animal an effective amount of a composition of the present invention. In one embodiment, the composition includes at least about 9% by weight protein, at least about 5% by weight fat, and at least about 0.05% by weight of at least one omega-3 polyunsaturated fatty acid. In a further embodiment said composition comprises at least one omega-3 polyunsaturated fatty acid chosen from docosahexaenoic acid (“DHA”) and eicosapentaenoic acid (“EPA”). In yet an additional embodiment, the composition further comprises at least one antioxidant and at least one nutrient chosen from choline, manganese, methionine, cysteine, L-carnitine, lysine, and mixtures thereof. In additional embodiments, the composition may comprise the components disclosed in Table 1 or Table 1A.

In another embodiment, the invention encompasses methods of measuring or characterizing the enhancement in the quality of life of an animal, particularly a senior or super senior animal, fed a composition described herein by quantitating the gene expression levels of one or more genes chosen from those disclosed in Tables 5-14 in said animal prior to and after feeding a composition disclosed herein and comparing said levels in the animal wherein an enhancement in the quality of life of said animal is reflected by a beneficial change in gene expression levels in said animal.

Another embodiment encompasses methods of altering the expression of at least one peptide in a mammal, the method comprising administering to the mammal a composition comprising at least about 9% by weight protein; at least about 5% by weight fat; and at least about 0.05% by weight of at least one omega-3 polyunsaturated fatty acid, wherein the at least one peptide is selected from the group consisting of X, Y and Z. With regard to the various embodiments presented herein, it is contemplated herein that the senior or super senior animal may be a senior or super senior large breed canine, regular breed canine, small breed canine or feline.

In another embodiment, the invention encompasses methods for screening one or more test compounds for its ability to alter the expression of at least one gene of interest in a mammal, the method comprising administering a control composition to a control group of mammals and determining the levels of expression of the at least one gene of interest, administering the one or more test compositions to an experimental group of mammals and determining the levels of expression of the least one gene of interest, wherein the test composition comprises at least about 9% by weight protein; at least about 5% by weight fat; and at least about 0.05% by weight of at least one omega-3 polyunsaturated fatty acid, and determining the differences in expression levels in the at least one gene of interest between the control and experimental groups of mammals after each group has been administered their respective compositions, wherein a difference in the expression levels of the at least one gene of interest indicates that the test composition is capable of altering the expression of the at least one gene of interest.

Another embodiment encompasses methods for screening one or more test compounds for its ability to alter the expression of at least one gene of interest in a mammal, the method comprising administering a control composition to a control group of mammals and determining the levels of expression of the at least one gene of interest, wherein the control composition comprises at least about 9% by weight protein; at least about 5% by weight fat; and at least about 0.05% by weight of at least one omega-3 polyunsaturated fatty acid, administering the one or more test compositions to an experimental group of mammals and determining the levels of expression of the least one gene of interest, and determining the differences in expression levels in the at least one gene of interest between the control and experimental groups of mammals after each group has been administered their respective compositions, wherein a difference in the expression levels of the at least one gene of interest indicates that the test composition is capable of altering the expression of the at least one gene of interest.

Other and further objects, features, and advantages of the present invention will be readily apparent to those skilled in the art.

### DETAILED DESCRIPTION OF THE INVENTION

### Definitions

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the advantageous methods, devices and materials are now described. All publications mentioned herein are incorporated by reference for the purpose of describing and disclosing the materials and methodologies that are reported in the publication which might be used in connection with the invention.

In practicing the present invention, many conventional techniques in molecular biology may be used. These techniques are well known and are explained in, for example, F. M. Ausubel, Ed. Current Protocols in Molecular Biology, Volumes I, II, and III, (Wiley, New York), 1997; J. Sambrook, E. F. Fritsch., T. Maniatis, Eds., Molecular Cloning: A Laboratory Manual (Cold Spring Harbor Laboratory, Cold Spring Harbor, N.Y., 1989).

As used herein and in the appended claims, the singular forms “a”, “an”, and “the” include plural reference unless the context clearly dictates otherwise.

The terms “senior” or “mature adult” refers to the life-stage of an animal. For small or regular breed canines, the “senior” life stage is about 7 to about 10 years of age. For felines, the “senior” life stage is about 7 to about 12 years of age. For large breed canines, over 5 years of age represents “super senior” as described below.

The terms “super senior” or “geriatric” refers to a specific life-stage of an animal. For small or regular breed canines, the super senior stage is any age greater than 10 years of age. For large breed canines, the super senior stage is any age greater than 5 years of age. For felines, the super senior stage is any age greater than 12 years of age.

The term “large breed” canine means a canine that normally weighs about 55 pounds or more when an adult.

The term “regular breed” canine means a canine that normally weighs less than about 55 pounds when an adult.

The term “small breed” canine means a canine that weighs less than about 20 pounds when an adult.

The term “super senior pet food composition” refers to any and all of the pet food compositions disclosed herein.

The term “carbohydrate” as used herein includes polysaccharides (e.g., starches and dextrins) and sugars (e.g. sucrose, lactose, maltose, glucose, and fructose) that are metabolized for energy when hydrolyzed. Examples of carbohydrates suitable for inclusion in the compositions disclosed herein include, but are not limited to, corn, grain sorghum, wheat, barley, and rice.

The term “antioxidant” means a substance that is capable of reacting with free radicals and neutralizing them. Illustrative examples of such substances include beta-carotene, selenium, coenzyme Q10 (ubiquinone), luetin, tocotrienols, soy isoflavones, S-adenosylmethionine, glutathione, taurine, N-acetylcysteine, vitamin E, vitamin C, lipoic acid and L-carnitine. Examples of foods containing useful levels of one or more antioxidants include but are not limited to ginkgo biloba, green tea, broccoli, citrus pulp, grape pomace, tomato pomace, carrot spinach, and a wide variety of fruit meals and vegetable meals. It will be understood by one of skill in the art that while units of antioxidants may be provided herein as “ppm”, appropriate amounts of antioxidants may also be provided as “IU/kg” where appropriate and customary for a given antioxidant such as, e.g., Vitamin E.

The terms “beneficial change” in gene expression, or gene expression may be “beneficially altered” and like terms refer to a modification in gene expression (e.g., up or down regulation of mRNA levels) such that levels of proteins or peptide chains encoded by the genes may be correspondingly modified such that an associated biological pathway may be more likely to function normally, such as in a healthy adult animal and with less tendency to reflect pathological changes in the pathway that, e.g., may be typical of a super senior or geriatric animal. Generally, beneficial changes in gene expression relate to improved health and/or reduced propensity for disease in an animal. As used herein, measuring differences in “gene expression” and like terms refer to, e.g., characterizing whether expression of a gene is up or down regulated in an animal compared to a control level. Gene expression levels can assessed by determining mRNA levels for a corresponding gene, or they may be inferred by determining protein or peptide chain levels. To be clear, determining “gene expression” or “gene expression levels” as used herein includes, but is not limited to, determining either corresponding RNA levels or peptide/protein levels or both. The invention is not limited to a particular method for determining protein or peptide or RNA levels, all of which are well known in the art. Moreover, gene expression and gene expression levels can be assessed in any cell or tissue that is appropriate for expression of the gene of interest. In one embodiment, gene expression is assessed in blood cells. In a more specific embodiment, the blood cells are lymphocytes. In an even more specific embodiment, the cells are T-lymphocytes. Other cell types include, but are not limited to, muscle cells, nerve cells, glial cells, endothelial cells, skin cells, liver cells, kidney cells, bone cells, other types of blood cells, such as but not limited to, macrophages. The cells may be primary cells, i.e., taken directly from an animal, such as cells isolated from recently drawn blood. The cells may also be non-primary, i.e. an established cell line through passage or even an immortalized cell line, such that the methods determining gene expression levels can be performed on established animal cell lines, e.g., CHO cells, prior to administration of a composition to an animal.

As used herein, a “gene” is a DNA molecule where at least a portion of which is transcribed into an RNA molecule. The DNA molecule may or may not include non-transcribed regions and/or non-translated regions, such as but not limited to introns, promoters, enhancer regions, 5′ untranslated regions.

The methods include the genes listed herein, as well as homologs. Thus, the methods of the present invention are not limited to the genes whose database accession numbers are disclosed herein and include homologs thereof. As used herein, a homolog of a gene listed herein means a gene whose coding or non-coding sequence may vary slightly from the reference sequence but also codes for the same or “equivalent” protein or peptide in a different organism. For example, the methods of the present invention relate to expression of phospholipase A2 in at least a canine A homolog of the canine phospholipase A2 gene would include, but would not be limited to, the feline phospholipase A2 gene, the bovine phospholipase A2 gene, the porcine phospholipase A2 gene, the equine phospholipase A2 gene and the primate phospholipase A2 gene. Homologs also include variations in the coding or non-coding sequences that account for slight variations across species. For example, the present invention relates to the human phospholipase A2 gene, and a homolog thereof would include, but would not be limited to a monkey or chimpanzee phospholipase A2 gene.

As used herein, “improving” or “enhancing” the quality of life of an animal refers to as an improvement or enhancement in one or more characteristics chosen from alertness, vitality, protection of cartilage, maintenance of muscle mass, digestibility, and skin and pelage quality. Additionally, improvement/enhancement in blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport are also contemplated.

An “improvement” or an “enhancement” in a characteristic or biological pathway refers to a modification in said characteristic or biological pathway such that there is a tendency for the characteristic or pathway to appear and/or function normally and with less tendency to reflect pathological changes in the characteristic or pathway that, e.g., may be typical of a super senior animal.

As used herein, methods to “treat” an animal suffering from a disease or disorder is also meant to encompass methods to prevent and/or to ameliorate the disease or disorder as well.

As used herein, “genes associated with the aging process” or “aging genes” or like terms refers to those genes which may be involved in the process of senescence in an animal. These genes may include, e.g., genes that encode for proteins that have a role in a number of biological functions such as inflammation, DNA repair or cell survival, fat or cholesterol metabolism, protein synthesis, immune regulation, cell growth and cell death.

Similarly, the “aging process”, as the term is used herein, refers to the process of senescence in an animal and may include changes in biological functions such as, e.g., inflammation, DNA repair or cell survival, fat or cholesterol metabolism, protein synthesis, cell growth and cell death.

As used herein, the phrase “modulating biological functions associated with the aging process” refers to op-regulating or down-regulating genes, which may be involved in the process of senescence in an animal. These genes may include, e.g., genes that encode for proteins that have a role in a number of biological functions such as inflammation, DNA repair or cell survival, fat or cholesterol metabolism, protein synthesis, immune regulation, cell growth and cell death.

### The Invention

The present invention encompasses compositions and methods for improving or enhancing the quality of life of a senior or super senior animal. The methods comprise feeding the animal a composition comprising at least about 9% by weight protein, at least about 5% by weight fat, and at least about 0.05% by weight omega-3 polyunsaturated fatty acid. The methods are useful for enhancing alertness, improving vitality, protecting cartilage, maintaining muscle mass, enhancing digestibility, and improving skin and pelage quality in a senior or super senior animal. The methods are also useful for improving in an animal one or more biological pathways chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and the electron transport pathway, such improvements also being reflected in overall beneficial changes at the genomic level. Methods for treating animals suffering from disorders or diseases associated with or related to these biological pathways comprising administering the compositions of the present invention are also contemplated herein.

Without being bound by theory, the benefits of the invention may be the result of physiological effects from the addition of omega-3 polyunsaturated fatty acids to a senior or super senior animal's diet. Similarly, the antioxidants, choline, and other nutrients may play a role in enhancing a senior or super senior animal's quality of life.

Although the methods of the present invention may improve an animal's quality of life by enhancing all of the above described characteristics or improving all of the described biological pathways, it is not necessary to demonstrate substantial improvements in each of the characteristics or pathways to achieve the “enhanced quality of life” as defined herein.

When the compositions are administered to a senior or super senior animal, the animal experiences an enhanced quality of life, e.g., exhibits or experiences one or more of enhanced alertness, improved vitality, protected cartilage, maintained muscle mass, enhanced digestibility, improved skin and pelage quality, as well as improvements in e.g., blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process and the electron transport pathway as indicated by overall beneficial changes at the genomic level. Methods for determining these measurements of quality of life are known to skilled artisans. For example, alertness can be measured by various means, including an analysis of metabolism and antioxidant markers, as well as through clinical studies with follow-up questions to participating pet owners. Potential metabolism markers may include ghrelin, GLP-1, thyroid hormone, and/or growth hormone. Potential markers of antioxidant status may include serum vitamin E, ORAC, glutathione peroxidase, alkanels, and/or cell damage indicators. Further, vitality can be measured by various means, including an analysis of metabolism and antioxidant markers, as well as through clinical studies with follow-up questions to participating pet owners. Similarly, cartilage protection can be measured by various means, including an analysis of arthritis biomarkers. Potential arthritis biomarkers may include type II collagen synthesis, matrix metaloproteinase, osteocalcin, alkaline phosphatase activity, COMP, and fragments of cartilage damage. Muscle mass maintenance can be measured by various means, including an analysis of body composition and digestibility can be measured by various means, including clinical studies with follow-up questions to participating pet owners and animal feeding to determine the percentage of nutrients digested Skin and pelage quality can be measured by various means, including clinical studies with follow-up questions to participating pet owners. Additionally, as discussed above, improvements in quality of life is also reflected at the genomic level, as evidenced by gene chip data which indicate beneficial changes on the expression of genes associated with various important biological pathways including blood clotting and platelet activation and aggregation, bone and muscle integrity, inflammatory responses, cartilage degradation and protection and pain response, DNA damage and repair pathways, neural function, glycogen synthesis and degradation, glycolysis, gluconeogenesis, the pentose phosphate pathway, the aging process, and the electron transport pathway. The identities of these genes are provided in the Examples below.

The methods of the invention are useful for enhancing the quality of life of humans and animals, including primates (e.g., monkeys, chimpanzees, etc.), companion animals (e.g., dogs, cats, horses, etc.), farm animals (e.g., goats, sheep, swine, cattle, etc.), laboratory animals (e.g., mice, rats, etc.), birds (e.g., domestic birds such as canaries, parrots, etc. and commercial birds such as chickens, ducks, turkeys, etc.), rodents (e.g., hamsters, guinea pigs, gerbils, rabbits, hedgehogs, ferrets, chinchillas, etc.), and wild, exotic, and zoo animals (e.g., wolves, bears, deer, etc.). In various embodiments, the animal is a cat, a dog, or a horse.

The compositions of the present invention are designed to enhance digestibility and improve chewability. Canine and feline foods are typically formulated based on life stage (age), size, body composition, and breed. Thus, some embodiments of the present invention include compositions that are formulated to address specific nutritional differences between regular or small breed dogs, large breed dogs, and cats.

The invention provides methods utilizing a variety of compositions containing at least one omega-3 polyunsaturated fatty acid. The compositions include foods, supplements, treats, and toys (typically chewable and consumable toys). The methods also provide the compositions to the designated animals over a period of time that is long enough to effectuate the improved quality of life. In one embodiment, the method provides the animal with a composition for at least thirty days.

The compositions for use in the methods of the present invention generally have an omega-3 polyunsaturated fatty acid content of at least about 0.02% (or about 0.05% to about 10%, or about 0.1% to about 6%) by weight on a dry matter basis. In some embodiments, the omega-3 polyunsaturated fatty acid is DHA. In other embodiments, the omega-3 polyunsaturated fatty acid is EPA. In still other embodiments, the omega-3 polyunsaturated fatty acid comprises a mixture of DHA and EPA.

In some embodiments, the composition containing omega-3 polyunsaturated fatty acid is a food. Although both liquid and solid foods are provided, solid foods are typically advantageous. Foods include both dry foods and wet foods. Some of the non-polyunsaturated fatty acid components of the food, and useful proportions, include those listed in Table 1.

TABLE 1

| | Proportion of the composition (% of dry weight of |
|---|---|
| Component | composition or parts per million) |
| Protein | about 9% to about 55%, or about 18% to about 30%, or |
| | about 33% to about 55% or about 18% to about 20% or |
| | about 33% to about 36% |
| Fat | about 7% to about 35%, or about 18% to about 35%, or |
| | about 7% to about 24%, or about 14% to about 24%, or |
| | about 14% to about 16% or about 18% to about 24% |
| Antioxidant | about 0 ppm to about 7500 ppm, or about 0.05 ppm to |
| | about 3600 ppm, or about 250 to about 3600, or about 250 |
| | ppm to about 1650 ppm, or about 5 ppm to about 225 ppm, |
| | or about 0.05 ppm to about 2.4 ppm |

In one embodiment, the methods of this invention comprise feeding a super senior animal a composition in an amount effective to enhance the animal's quality of life. Such compositions generally comprise:
(a) 0.02% (or about 0.05% to about 10%, or about 0.1% to about 6%) of at least one omega-3 polyunsaturated fatty acid, and
(b) at least one of the following:
   (i) about 10% to about 55% (or about 18% to about 30%, or about 33% to about 55% or about 18% to about 20% or about 33% to about 36%) protein,
   (ii) about 7% to about 35% (or about 18% to about 35%, or about 7% to about 24%, or about 14% to about 24%, or about 14% to about 16% or about 18% to about 24%) fat, and
   (iii) at least about 0.05 (or about 0.05 ppm or IU/kg to about 7500 ppm or IU/kg, or about 250 ppm or IU/kg to about 3600 ppm or IU/kg, or about 250 ppm or IU/kg to about 1650 ppm or IU/kg, or about 5 ppm or IU/kg to about 225 ppm or IU/kg, or about 0.05 ppm or IU/kg to about 2.4 ppm or IU/kg) antioxidant.

In another embodiment, the methods of this invention comprise feeding a super senior regular or small breed canine a composition in an amount effective to enhance the canine's quality of life. The composition generally comprises:
(a) at least one of the following:
   (i) at least about 0.02% (or about 0.02% to about 0.3%, or about 0.05% to about 0.3%, or about 0.05% to about 0.2%) DHA, and
   (ii) at least about 0.1% (or about 0.1% to about 0.5%, or about 0.2% to about 0.5%, or about 0.2% to about 0.3%) EPA,
(b) at least about 9% (or about 9% to about 30%, or about 18% to about 30%, or about 18% to about 20%) protein,
(c) at least about 7% (or about 7% to about 24%, or about 14% to about 24%, or about 14% to about 16%) fat, and
(d) at least one of the following:
   (i) at least about 250 IU/kg (or about 250 IU/kg to about 1500 IU/kg, or about 500 IU/kg to about 1500 IU/kg, or about 500 IU/kg to about 1000 IU/kg) vitamin E,
   (iv) at least about 50 ppm (or about 50 ppm to about 500 ppm, or about 100 ppm to about 500 ppm, or about 100 ppm to about 301 ppm) vitamin C,
   (v) at least about 600 ppm (or about 600 ppm to about 2400 ppm, or about 1260 ppm to about 2400 ppm, or about 1260 ppm to about 1545 ppm) taurine,
   (vi) at least about 50 ppm (or about 50 ppm to about 200 ppm, or about 100 to about 160, or about 100 to about 155) lipoic acid, and
   (vii) at least about 50 ppm (or about 50 ppm to about 500 ppm, or about 200 ppm to about 500 ppm, or about 200 ppm to about 350 ppm) carnitine.

In another embodiment, the methods of this invention comprise feeding a super senior large breed canine a composition in an amount effective to enhance the canine's quality of life. The compositions generally comprise:
(a) at least one of the following:
   (i) at least about 0.02% (or about 0.02% to about 0.3%, or about 0.05% to about 0.3%, or about 0.05% to about 0.2%) DHA, and
   (ii) at least about 0.1% (or about 0.1% to about 0.5%, or about 0.2% to about 0.5%, or about 0.2% to about 0.3%) EPA,
(b) at least about 9% (or about 9% to about 30%, or about 18% to about 30%, or about 18% to about 20%) protein,
(c) at least about 7% (or about 7% to about 24%, or about 14% to about 24%, or about 14% to about 16%) fat, and
(d) at least one of the following:
   (i) at least about 250 IU/kg (or about 250 IU/kg to about 1500 IU/kg, or about 500 IU/kg to about 1500 IU/kg, or about 500 IU/kg to about 1000 IU/kg) vitamin E,
   (viii) at least about 50 ppm (or about 50 ppm to about 500 ppm, or about 100 ppm to about 500 ppm, or about 100 ppm to about 301 ppm) vitamin C,
   (ix) at least about 600 ppm (or about 600 ppm to about 2400 ppm, or about 1260 ppm to about 2400 ppm, or about 1260 ppm to about 1575 ppm) taurine, and
   (x) at least about 50 ppm (or about 50 ppm to about 200 ppm, or about 100 to about 160, or about 100 to about 155) lipoic acid, and
   (xi) at least about 50 ppm (or about 50 ppm to about 500 ppm, or about 200 ppm to about 500 ppm, or about 200 ppm to about 350 ppm) carnitine.

In another embodiment, the methods of this invention comprise feeding a super senior feline a composition in an amount effective to enhance the feline's quality of life. The compositions generally comprise:
(a) at least one of the following:
   (i) at least about 0.05% (or about 0.05% to about 0.30%, or about 0.1% to about 0.30%, or about 0.1% to about 0.2%) DHA, and
   (ii) at least about 0.1% (or about 0.1% to about 0.5%, or about 0.2% to about 0.5%, or about 0.2% to about 0.3%) EPA,
(b) at least about 15% (or about 15% to about 55%, or about 30% to about 55%, or about 33% to about 36%) protein,
(c) at least about 9% (or about 9% to about 35%, or about 18% to about 35%, or about 18% to about 24%) fat, and
(d) at least one of the following:
   (i) at least about 250 IU/kg (or about 250 IU/kg to about 1500 IU/kg, or about 500 IU/kg to about 1500 IU/kg, or about 500 IU/kg to about 1100 IU/kg) vitamin E,
   (xii) at least about 50 ppm (or about 50 ppm to about 300 ppm, or about 100 ppm to about 300 ppm, or about 100 ppm to about 200 ppm) vitamin C,
   (xiii) at least about 1100 ppm (or about 1100 ppm to about 3500 ppm, or about 2300 ppm to about 3500 ppm, or about 2300 ppm to about 2350 ppm) taurine, and
   (xiv) at least about 200 ppm (or about 200 to about 750 ppm, or about 400 ppm to about 750 ppm, or about 400 to about 525 ppm) carnitine, and
   (xv) at least about 0.05% (or about 0.05% to about 0.6%, or about 0.1% to about 0.6%, or about 0.1% to about 0.4%) cystine.

In another embodiment, the methods of this invention comprise feeding a super senior animal a composition in an amount effective to enhance the animal's alertness and vitality. The composition generally comprises:
(a) 0.02% (or about 0.05% to about 10%, or about 0.1% to about 6%) at least one omega-3 polyunsaturated fatty acid, and
(b) at least one of the following:
   (xvi) about 10% to about 55% (or about 18% to about 30%, or about 33% to about 55% or about 18% to about 20% or about 33% to about 36%) protein,
   (xvii) about 7% to about 35% (or about 18% to about 35%, or about 7% to about 24%, or about 14% to about 24%, or about 14% to about 16% or about 18% to about 24%) fat,
   (xviii) at least about 0.05 (or about 0.05 ppm to about 7500 ppm, or about 250 to about 3600, or about 250 ppm to about 1650 ppm, or about 5 ppm to about 225 ppm, or about 0.05 ppm to about 2.4 ppm) antioxidant, and
   (xix) at least about 1000 ppm (or about 1000 ppm to about 5000 ppm, about 3300 ppm to about 5000 ppm, or about 2000 ppm to about 3000 ppm, or about 3000 ppm to about 4000 ppm) choline.

In another embodiment, the methods of this invention comprise feeding a super senior regular or small breed canine a composition in an amount effective to enhance the canine's alertness and vitality. The composition generally comprises:
(a) at least one of the following:
   (i) at least about 0.02% (or about 0.02% to about 0.3%, or about 0.05% to about 0.3%, or about 0.05% to about 0.2%) DHA, and (ii) at least about 0.1% (or about 0.1% to about 0.5%, or about 0.2% to about 0.5%, or about 0.2% to about 0.3%) EPA,
(b) at least about 9% (or about 9% to about 30%, or about 18% to about 30%, or about 18% to about 20%) protein,
(c) at least about 7% (or about 7% to about 24%, or about 14% to about 24%, or about 14% to about 16%) fat,
(d) at least one of the following:
   (i) at least about 250 IU/kg (or about 250 IU/kg to about 1500 IU/kg, or about 500 IU/kg to about 1500 IU/kg, or about 500 IU/kg to about 1000 IU/kg) vitamin E,
   (xx) at least about 50 ppm (or about 50 ppm to about 500 ppm, or about 100 ppm to about 500 ppm, or about 100 ppm to about 301 ppm) vitamin C,
   (xxi) at least about 600 ppm (or about 600 ppm to about 2400 ppm, or about 1260 ppm to about 2400 ppm, or about 1260 ppm to about 1545 ppm) taurine, and
   (xxii) at least about 50 ppm (or about 50 ppm to about 200 ppm, or about 100 to about 160, or about 100 to about 155) lipoic acid, and
   (xxiii) at least about 50 ppm (or about 50 ppm to about 500 ppm, or about 200 ppm to about 500 ppm, or about 200 ppm to about 350 ppm) carnitine,
(e) at least about 1000 ppm (or about 1000 ppm to about 3200 ppm, or about 2000 ppm to about 3200 ppm, or about 2000 ppm to about 2500 ppm) choline,
(f) at least about 50 ppm (or about 50 ppm to about 150 ppm, or about 100 ppm to about 150 ppm, or about 100 ppm to about 110 ppm) manganese, and
(g) at least about 0.4% (or about 0.4% to about 2%, or about 0.9% to about 2%, or about 0.9% to about 1.2%) lysine, and
(h) at least about 0.4% to about 1.5% methionine.

In another embodiment, the methods of this invention comprise feeding a super senior large breed canine a composition in an amount effective to enhance the canine's alertness and vitality. The composition generally comprises:
(a) at least one of the following:
   (i) at least about 0.02% (or about 0.02% to about 0.3%, or about 0.05% to about 0.3%, or about 0.05% to about 0.2%) DHA, and
   (ii) at least about 0.1% (or about 0.1% to about 0.5%, or about 0.2% to about 0.5%, or about 0.2% to about 0.3%) EPA,
(b) at least about 9% (or about 9% to about 30%, or about 18% to about 30%, or about 18% to about 20%) protein,
(c) at least about 7% (or about 7% to about 24%, or about 14% to about 24%, or about 14% to about 16%) fat,
(d) at least one of the following:
   (i) at least about 250 IU/kg (or about 250 IU/kg to about 1500 IU/kg, or about 500 IU/kg to about 1500 IU/kg, or about 500 IU/kg to about 1000 IU/kg) vitamin E,
   (xxiv) at least about 50 ppm (or about 50 ppm to about 500 ppm, or about 100 ppm to about 500 ppm, or about 100 ppm to about 301 ppm) vitamin C,
   (xxv) at least about 600 ppm (or about 600 ppm to about 2400 ppm, or about 1260 ppm to about 2400 ppm, or about 1260 ppm to about 1575 ppm) taurine, and
   (xxvi) at least about 50 ppm (or about 50 ppm to about 200 ppm, or about 100 to about 160, or about 100 to about 155) lipoic acid, and
   (xxvii) at least about 50 ppm (or about 50 ppm to about 500 ppm, or about 200 ppm to about 500 ppm, or about 200 ppm to about 350 ppm) carnitine,
(e) at least about 1000 ppm (or about 1000 ppm to about 3200 ppm, or about 2000 ppm to about 3200 ppm, or about 2000 ppm to about 2500 ppm) choline,
(f) at least about 50 ppm (or about 50 ppm to about 150 ppm, or about 100 ppm to about 150 ppm, or about 100 ppm to about 110 ppm) manganese, and
(g) at least about 0.4% (or about 0.4% to about 2%, or about 0.9% to about 2%, or about 0.9% to about 1.2%) lysine, and
(h) at least about 0.4% to about 1.5% methionine.

In another embodiment, the methods of this invention comprise feeding a super senior feline a composition in an amount effective to enhance the feline's alertness and vitality. The composition generally comprises:
(a) at least one of the following:
   (i) at least about 0.05% (or about 0.05% to about 0.30%, or about 0.1% to about 0.30%, or about 0.1% to about 0.2%) DHA, and
   (ii) at least about 0.1% (or about 0.1% to about 0.5%, or about 0.2% to about 0.5%, or about 0.2% to about 0.3%) EPA,
(b) at least about 15% (or about 15% to about 55%, or about 30% to about 55%, or about 33% to about 36%) protein,
(c) at least about 9% (or about 9% to about 35%, or about 18% to about 35%, or about 18% to about 24%) fat,
(d) at least one of the following:
   (i) at least about 250 IU/kg (or about 250 IU/kg to about 1500 IU/kg, or about 500 IU/kg to about 1500 IU/kg, or about 500 IU/kg to about 1100 IU/kg) vitamin E,
   (xxviii) at least about 50 ppm (or about 50 ppm to about 300 ppm, or about 100 ppm to about 300 ppm, or about 100 ppm to about 200 ppm) vitamin C,
   (xxix) at least about 1100 ppm (or about 1100 ppm to about 3500 ppm, or about 2300 ppm to about 3500 ppm, or about 2300 ppm to about 2350 ppm) taurine, and
   (xxx) at least about 200 ppm (or about 200 to about 750 ppm, or about 400 ppm to about 750 ppm, or about 400 to about 525 ppm) carnitine, and
   (xxxi) at least about 0.05% (or about 0.05% to about 0.6%, or about 0.1% to about 0.6%, or about 0.1% to about 0.4%) cystine,
(e) at least about 1600 ppm (or about 1600 ppm to about 5000 ppm, or about 3300 ppm to about 5000 ppm, or about 3300 ppm to about 3400 ppm) choline,
(f) at least about 50 ppm (or about 50 ppm to about 150 ppm, or about 100 ppm to about 150 ppm, or about 100 ppm to about 110 ppm) manganese, and
(g) at least about 0.7% (or about 0.7% to about 3%, or about 1.4% to about 3%, or about 1.4% to about 1.7%) lysine, and
(h) at least about 0.4% to about 1.5% methionine.

In another embodiment, this invention provides a method for improving the quality of life of a senior or super senior small or regular breed canine The method comprises feeding the canine a composition comprising:
about 60% to about 70% by weight carbohydrate;
about 15% to about 25% by weight protein chosen from animal protein and vegetable protein;
about 5% to about 7% by weight fat chosen from animal fat and vegetable fat;
about 2.5% to about 4% by weight of at least one omega-3 polyunsaturated fatty acids;
about 1% to about 4% by weight fiber;
about 1% to about 2% by weight minerals; and
about 0.5 to about 1.5% by weight vitamins.

In another embodiment, this invention provides a method for improving the quality of life of a senior or super senior large breed canine. The method comprises feeding the canine a composition comprising:
about 60% to about 70% by weight carbohydrate;
about 15% to about 25% by weight protein chosen from animal protein and vegetable protein;
about 5% to 10% by weight fat chosen from animal fat and vegetable fat;
about 3% to about 5% by weight of at least one omega-3 polyunsaturated fatty acids;
about 1% to about 4% by weight fiber;
about 0.5% to about 1% by weight minerals; and
about 0.75 to about 1.25% by weight vitamins.

In another embodiment, this invention provides a method for improving the quality of life of a senior or super senior feline. The method comprises feeding the feline a composition comprising:
about 30% to about 35% by weight carbohydrate;
about 35% to about 50% by weight protein chosen from animal protein and vegetable protein;
about 12% to about 15% by weight fat chosen from animal fat and vegetable fat;
about 1% to about 2% by weight of at least one omega-3 polyunsaturated fatty acids;
about 1% to about 5% by weight fiber;
about 1% to about 2% by weight minerals; and
about 1% to about 2% by weight vitamins.

In a further embodiment, this invention provides a method for improving the quality of life of a senior or super senior animal comprising feeding the animal (e.g., small, regular or large breed canine or feline, as the case may be) a composition comprising the components as indicated in Table 1A below:

TABLE 1A
Chemical composition of Super Senior
Foods

| | Small/Regular | | |
|---|---|---|---|
| | Breed | Large Breed | |
| Nutrient Component | Canine | Canine | Feline |
| Crude Protein, % | 20.1 | 19.34 | 35.73 |
| Fat, % | 16.45 | 16.92 | 22.47 |
| Calcium, % | 0.71 | 0.73 | 0.94 |
| Phosphorus, % | 0.61 | 0.68 | 0.77 |
| EPA, % | 0.32 | 0.32 | 0.23 |
| DHA, % | 0.22 | 0.22 | 0.32 |
| Linoleic Acid, % | 3.96 | 4.04 | 5.05 |
| Total N-3 fatty acids, % | 1.3 | 2.24 | 1.14 |
| Total N-6 fatty acids, % | 3.96 | 3.99 | 5.09 |
| Taurine, ppm | 1400 | 15.25 | 2100 |
| Carnitine, ppm | 314 | 337 | 367 |
| Methioinine, % | 1 | 1.19 | 1.32 |
| Cystine, % | 0.25 | 0.24 | 0.47 |
| Manganese, ppm | 87 | 100 | 104 |
| Vitamin E, IU/kg | 1492 | 1525 | 1292 |
| Vitamin C, ppm | 127 | 261 | 141 |
| Lipoic Acid, ppm* | 101 | 135 | |

*Lipoic acid based on formulated, not analyzed values.

The compositions for use in the methods of this invention further comprise at least one nutrient chosen from manganese, methionine, cysteine, mixtures of methionine and cysteine, L-carnitine, lysine, and arginine. Specific advantageous amounts for each component in a composition will depend on a variety of factors including, for example, the species of animal consuming the composition; the particular components included in the composition; the age, weight, general health, sex, and diet of the animal; the animal's consumption rate, and the like. Thus, the component amounts may vary widely, and may even deviate from the proportions given herein.

The omega-3 fatty acids may be obtained from a variety of sources. One convenient source is fish oils from, for example, menhaden, mackerel, herring, anchovy, and salmon. DHA and EPA are typical fatty acids present in such fish oils, and, together often make up a significant portion of the oil, such as about 25% to about 38% of the oil.

When the composition is an animal food, vitamins and minerals preferably are included in amounts required to avoid deficiency and maintain health. These amounts are readily available in the art. The National Research Council (NRC), for example, provides recommended amounts of such ingredients for farm animals. See, e.g., Nutrient Requirements of Swine (10th Rev. Ed., Nat'l Academy Press, Wash. D.C., 197298), Nutrient Requirements of Poultry (9th Rev. Ed., Nat'l Academy Press, Wash. D.C., 1994), Nutrient Requirements of Horses (Fifth Rev. Ed., Nat'l Academy Press, Wash. D.C., 1989), Nutrient Requirements of Dogs and Cats (Nat'l Academy Press, Wash. D.C., 2006). The American Feed Control Officials (AAFCO), for example, provides recommended amounts of such ingredients for dogs and cats. See American Feed Control Officials, Inc., Official publication, pp. 126-140 (2003). Examples of vitamins useful as food additives include vitamin A, B1, B2, B6, B12, C, D, E, K, H (biotin), K, folic acid, inositol, niacin, and pantothenic acid. Examples of minerals and trace elements useful as food additives include calcium, phosphorus, sodium, potassium, magnesium, copper, zinc, chloride, and iron salts.

The methods of the present invention include compositions that may further contain other additives known in the art. Preferably, such additives are present in amounts that do not impair the purpose and effect provided by the invention. Examples of additives include, for example, substances with a stabilizing effect, processing aids, substances that enhance palatability, coloring substances, and substances that provide nutritional benefits.

Stabilizing substances include, for example, substances that tend to increase the shelf life of the composition. Potentially suitable examples of such substances include, for example, preservatives, antioxidants, synergists and sequestrants, packaging gases, stabilizers, emulsifiers, thickeners, gelling agents, and humectants. Examples of emulsifiers and/or thickening agents include, for example, gelatin, cellulose ethers, starch, starch esters, starch ethers, and modified starches.

Additives for coloring, palatability (“pal enhancers”), and nutritional purposes include, for example, colorants (e.g., iron oxide, such as the red, yellow, or brown forms); sodium chloride, potassium citrate, potassium chloride, and other edible salts; vitamins; minerals; and flavoring. Such additives are known in the art. See, e.g., U.S. Pat. No. 3,202,514. See also, U.S. Pat. No. 4,997,671. Flavorants include, for example, dairy product flavorants (e.g., milk or cheese), meat flavorants (e.g., bacon, liver, beef, poultry, or fish), oleoresin, pinacol, and the various flavorants identified in the trade by a FEMA (Flavor Extract Manufacturers Association) number. Flavorants help provide additional palatability, and are known in the art. See, e.g., U.S. Pat. No. 4,997,672. See also, U.S. Pat. No. 5,004,624. See also, U.S. Pat. No. 5,114,704. See also, U.S. Pat. No. 5,532,010. See also, U.S. Pat. No. 6,379,727. The concentration of such additives in the composition typically may be up to about 5% by weight. In some embodiments, the concentration of such additives (particularly where such additives are primarily nutritional balancing agents, such as vitamins and minerals) is about 0% to about 2.0% by weight. In some embodiments, the concentration of such additives (again, particularly where such additives are primarily nutritional balancing agents) is about 0% to about 1.0% by weight.

Supplements include, for example, a feed used with another feed to improve the nutritive balance or performance of the total. Supplements include compositions that are fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Inc. Official Publication, p. 220 (2003). Supplements may be in various forms including, for example, powders, liquids, syrups, pills, encapsulated compositions, and the like.

Treats include, for example, compositions that are given to an animal to entice the animal to eat during a non-meal time. Treats for canines include, for example, dog bones. Treats may be nutritional, wherein the composition comprises one or more nutrients, and may, for example, have a composition as described above for food. Non-nutritional treats encompass any other treats that are non-toxic.

Toys include, for example, chewable toys. Toys for dogs include, for example, artificial bones. There is a wide range of suitable toys currently marketed. See, e.g., U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). The invention provides both partially consumable toys (e.g., toys comprising plastic components) and fully consumable toys (e.g., rawhides and various artificial bones). It should be further recognized that this invention provides toys for both human and non-human use, particularly for companion, farm, and zoo animal use, and particularly for dog, cat, or bird use.

A “food” is a nutritionally complete diet for the intended recipient animal (e.g., domestic cat or domestic dog). A “nutritionally complete diet” is a diet that includes sufficient nutrients for maintenance of normal health of a healthy animal on the diet. The methods of this invention utilize compositions that are not intended to be restricted by any specific listing of proteinaceous or fat ingredients or product form. The compositions can be prepared in, for example, a dry, canned, wet, or intermediate moisture form using conventional pet food processes. In some embodiments, the moisture content is about 10% to about 90% of the total weight of the composition. In other embodiments, the moisture content is about 65% to about 75% of the total weight of the composition.

In preparing a composition for use with the methods of the present invention, any ingredient (e.g., fish oil) generally may, for example, be incorporated into the composition during the processing of the formulation, such as during and/or after mixing of other components of the composition. Distribution of these components into the composition can be accomplished by conventional means. In one embodiment, ground animal and poultry proteinaceous tissues are mixed with the other ingredients, including fish oils, cereal grains, other nutritionally balancing ingredients, special-purpose additives (e.g., vitamin and mineral mixtures, inorganic salts, cellulose and beet pulp, bulking agents, and the like); and water that is sufficient for processing is also added. These ingredients preferably are mixed in a vessel suitable for heating while blending the components. Heating of the mixture may be effected using any suitable manner, such as, for example, by direct steam injection or by using a vessel fitted with a heat exchanger. Following the addition of the last ingredient, the mixture is heated to a temperature range of about 50° F. (10° C.) to about 212° F. (100° C.). In some embodiments, the mixture is heated to a temperature range of about 70° F. (21° C.) to about 140° F. (60° C.). Temperatures outside these ranges are generally acceptable, but may be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. (110° C.) for an appropriate time, which is dependent on, for example, the temperature used and the composition.

Methods of the present invention include utilizing compositions that can be prepared in a dry form using conventional processes. In one embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing.

The compositions are also designed to be easier to chew. Canine and feline foods are typically formulated based on life stage (age), size, body composition, and breed. In the methods of this invention, some embodiments of the compositions address specific nutritional differences between super senior regular or small breed dogs, large breed dogs, and cats.

All percentages expressed herein are on a weight by dry matter basis unless specifically stated otherwise.

As noted previously, this invention is directed, in part, to a method for enhancing the quality of life of an animal. The method comprises feeding a senior or super senior animal a composition in an amount effective to enhance alertness, improve vitality, protect cartilage, maintain muscle mass, enhance digestibility, and improve skin and pelage quality. Additionally, we now report herein our surprising discovery that the enhanced quality of life of an animal achieved by administration of the compositions of the present invention is reflected at the genomic level. While it may be that a change in expression of any one gene disclosed in the tables presented below may result in beneficial or deleterious biological effects, the data presented herein indicate that, overall, the observed expression profiles are consistent with the beneficial biological effects seen in vivo after administration of the diets disclosed herein. Specifically, gene chip data indicate that the expression of genes that encode proteins associated with or related to several biological pathways such as blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport are, for the most part, beneficially altered through administration to the animal of compositions described herein. Thus, the invention also relates to methods of measuring or characterizing the enhancement in the quality of life of an animal, particularly a senior or super senior animal, fed a composition described herein by quantitating the gene expression levels of one or more genes chosen from those disclosed in Tables 5-14 in said animal prior to and after feeding a composition disclosed herein and comparing said levels in the animal wherein an enhancement in the quality of life of said animal is reflected by a beneficial change in gene expression levels in said animal.

Quantitation of gene expression may be carried out in numerous ways familiar to one of skill in the art and include such techniques as RT PCR as well as gene chip assays and Northern blotting. Thus, it is contemplated herein that the expression levels detected may be used, for example, in methods to measure enhancement in the quality of life of an animal as disclosed herein.

There are certain age-induced changes in gene expression patterns (see, for example, P. Tollet-Egnell et al., Molecular Endocrinology, 15(2):308-318 (2001)). Without being bound by theory, such changes in gene expression patterns may be related to senescence, the aging mechanism. C-K Lee et al., Science, 285:1390-1393 (1999) reported that alterations in the gene expression profile of the aging process in mice can be completely or partially prevented by caloric restriction. We have found that, surprisingly, the changes in expression of certain genes as an animal, such as a dog, ages from a healthy adult animal to a geriatric animal can be reversed by a diet of super senior dog food according to the present invention. Thus, comparing the gene expression pattern in a healthy adult dog to the gene expression pattern in a geriatric dog, one finds certain genes expressed higher (“up”) in the geriatric dog while other genes are expressed lower (“down”). Surprisingly, we have found that by feeding a diet of super senior dog food according to the present invention to a geriatric dog, the gene expression pattern can be reversed. That is, comparing the gene expression pattern in a geriatric dog fed a control diet to the gene expression pattern in a geriatric dog fed a diet of super senior dog food of the present invention, one finds that certain genes are expressed higher (“up”) under the control dog food regimen, while other genes are expressed lower (“down”) under the control dog food regimen. The result is that the geriatric dogs under the super senior dog food diet of the present invention had their gene expression profiles altered towards that of healthy adult dogs. Comparing the list of genes that correlate in the opposite sense to the healthy adult dog/geriatric dog expression pattern, we found genes provided in Tables 15-20 below that surprisingly demonstrate that the super senior dog food of the present invention can reverse the alteration in expression that certain genes undergo as a part of the aging process. Thus, the quality of life of geriatric animals can be benefited by modifying the aging process in that the gene expression pattern of certain genes are altered towards that of a healthy adult dog from the pattern of a geriatric dog.

Accordingly, this invention is directed, in part, to a method for enhancing the quality of life of an animal comprising feeding a senior or super senior animal a composition in an amount effective to alter the gene expression pattern of certain genes (provided on Tables 15-20 where the direction of adult vs geriatric is the same as the direction of super senior vs control) towards the pattern of a healthy adult dog form the pattern of a geriatric dog. The method enhances the quality of life of an animal by modifying the expression of genes associated with the aging process such that the gene expression pattern is altered towards that of a healthy adult animal from that of a geriatric animal.

In one aspect, this invention is directed to a method for improving the quality of life of a senior or super senior animal comprising feeding the animal a composition comprising at least about 9% by weight protein; at least about 5% by weight fat; and at least about 0.05% by weight of at least one omega-3 polyunsaturated fatty acid, wherein the method comprises feeding the animal the composition in an amount effective to enhance the animal's quality of life, wherein enhanced quality of life is evidenced by a change in expression of one or more genes which encode proteins associated with the aging process. As described herein, these genes are generally referred to as genes associated with the aging process, however, it should be noted that these genes specifically may be related to biological pathways chosen from, e.g., inflammation, DNA repair, cell survival, fat or cholesterol metabolism, immune regulation, protein synthesis, cell growth and cell death.

In an embodiment of this aspect, the change in expression is of one or more genes listed on Tables 15-19 and wherein the change in expression is towards the expression level in a healthy adult animal as compared to the expression level in a geriatric animal.

In another embodiment of this aspect, the animal is a dog.

In another aspect, this invention is directed to a method for improving the quality of life of a senior or super senior animal comprising feeding the animal a composition comprising at least about 9% by weight protein; at least about 5% by weight fat; and at least about 0.05% by weight of at least one omega-3 polyunsaturated fatty acid, wherein the method comprises feeding the animal the composition in an amount effective to enhance the animal's quality of life, wherein enhanced quality of life is evidenced by a change in expression of one or more genes listed on Table 20 and wherein the change in expression is towards the expression level in a healthy adult animal as compared to the expression level in a geriatric animal.

In an embodiment of this aspect, the animal is a dog.

It is also contemplated herein that the invention relates to methods for treating an animal suffering from disorders or disease associated with or relating to any one of more of the following biological pathways: blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport comprising administering to the animal an effective amount of a food composition of the present invention.

This invention is not limited to the particular methodology, protocols, and reagents described herein because they may vary. Further, the terminology used herein is for the purpose of describing particular embodiments only and is not intended to limit the scope of the present invention. The terms “comprise”, “comprises”, and “comprising” are to be interpreted inclusively rather than exclusively.

Unless defined otherwise, all technical and scientific terms and any acronyms used herein have the same meanings as commonly understood by one of ordinary skill in the art in the field of the invention. Many modifications and variations of the present invention are possible in light of the above teachings. It is therefore to be understood that within the scope of the appended claims the invention may be practiced otherwise than as specifically described.

All patents, patent applications, and publications mentioned herein are incorporated herein by reference in their entirety. However, where there is a conflict between a definition in the present disclosure and that of a cited reference, the present disclosure controls.

### EXAMPLES

This invention can be further illustrated by the following examples, although it will be understood that these examples are included merely for purposes of illustration and are not intended to limit the scope of the invention unless otherwise specifically indicated.

### Example 1

A composition formulated for senior or super senior regular or small breed canines is described in Table 2.

TABLE 2
Ingredient Composition for Canine Regular or Small Breed
Super Senior

| Ingredient | % of composition |
|---|---|
| Carbohydrate | 65.83 |
| Animal Protein | 14.31 |
| Vegetable Protein | 6.05 |
| Animal/Vegetable Fat | 6.60 |
| Omega Fat | 3.38 |
| Fiber | 1.42 |
| Minerals | 1.63 |
| Vitamins | 0.78 |

### Example 2

A composition formulated for senior or super senior large breed canines is described in Table 3.

TABLE 3
Ingredient Composition for Canine Large Breed Super Senior

| Ingredient | % of composition |
|---|---|
| Carbohydrate | 65.15 |
| Animal Protein | 14.79 |
| Vegetable Protein | 6.45 |
| Animal/Vegetable Fat | 6.23 |
| Omega Fat | 4.12 |
| Fiber | 1.30 |
| Minerals | 0.91 |
| Vitamins | 1.05 |

### Example 3

A composition formulated for senior or super senior felines is described in Table 4.

TABLE 4
Ingredient Composition for Feline Super Senior

| Ingredient | % of composition |
|---|---|
| Carbohydrate | 31.47 |
| Animal Protein | 25.57 |
| Vegetable Protein | 20.14 |
| Animal/Vegetable Fat | 13.31 |
| Omega Fat | 1.61 |
| Fiber | 4.80 |
| Minerals | 1.77 |
| Vitamins | 1.34 |

### Example 4

### Genomic Analysis of Control vs. Super Senior Pet Food

To further characterize the nutritional benefits of the super senior pet food compositions of the present invention, gene expression profiles from animals fed the compositions compared to control animals are assayed and the results are described in detail below.

### Materials and Methods:

### Study Design:

Blood samples are drawn from 9 Beagles according to conventional methods before and after feeding for 14 days on Super Senior K9 diet (a total of 18 samples). Each sample taken after the 14-day trial is compared to its own control.

Isolation of Lymphocytes from Canine Blood

### Reagents:

4 ml canine blood, heparin or EDTA tubes, Hank's Balanced Salt Solution (Gibco 14175-095), HEPES buffer (Gibco 15630-080), Accu-Paque (Accurate Chemical & Scientific Corp AN3100).

### Materials/Equipment:

Transfer pipettes (VWR 14670-147), 14 ml centrifuge tubes w/ caps, 9″ Pasteur pipettes, 1.5 ml microcentrifuge tubes (VWR 20170-038), centrifuge tube racks, microcentrifuge tube bale, waste container, Beckman Coulter Allegra 25R Centrifuge, SN AJC01J015Eppendorf Centrifuge, 5417C.

### Solutions:

Hank's Balanced Salt Solution (HBSS) w/25 mM HEPES buffer solution is made by adding 12.8 ml of HEPES buffer solution to a 500 ml bottle of HBSS. Hank's Balanced Salt Solution and Accu-Paque need to be removed from the refrigerator and placed at room temperature at least 30 minutes before beginning the lymphocyte isolation. Both solutions should be place back in the refrigerator (4° C.) immediately following their use.

Procedure:

1. Measure 4 ml of HBSS w/ HEPES into the correct number of 14 ml centrifuge tubes (one tube for each 4 ml draw of blood)

2. Using a transfer pipette, transfer 4 ml blood from the Vacutainer® tubes to the 14 ml centrifuge tube containing the HBSS w/ HEPES.

3. Mix the sample well using the transfer pipette to pipette up and down for 30 seconds.

4. Insert a 9″ Pasteur pipette into each of the 14 ml centrifuge tubes. Make sure the bottom tip of the Pasteur pipette touches the bottom of the tube.

5. Using a transfer pipette, slowly add 4 ml of Accu-Paque by running the liquid down the inside of the Pasteur pipette allowing gravity to layer the Accu-Paque under the diluted blood sample.

6. Plug the top of the Pasteur pipette using your finger and gently remove the pipette.

7. Centrifuge the tubes at 800×g for 20 minutes at room temperature. For puppy blood a longer centrifugation of 45 minutes is necessary to allow for a good separation of RBC's from WBC's.

8. Using a transfer pipette, carefully remove the top layer to within 0.5 cm of the middle opaque layer and discard.

9. Using a new transfer pipette, carefully remove the middle opaque layer and transfer to a 1.5 ml microcentrifuge tube. Be careful not to transfer any of the bottom layers.

10. Centrifuge the microcentrifuge tubes at 13,200 rpm for 3.5 minutes at room temperature.

11. Carefully remove the supernatant and flash freeze the remaining pellet (lymphocytes) in liquid nitrogen. Store the final samples at −80° C.

### RNA Isolation:

### Reagents:

Deionized H₂O, Absolute ethanol (Sigma E7023), RNA Storage Solution (Ambion 7000), RNase Zap® (Ambion 9780), Buffer RLT, Buffer RW1 and Buffer RPE (provided in the RNeasy Mini Kit).

### Equipment/Materials:

RNeasy Mini Kit (Qiagen 74104), QIAshredder spin columns (Qiagen 79656), P1000 Pipetman pipette (Rainin), P200 Pipetman pipette (Rainin), 100-100 μl filtered pipette tips (USA Scientific 1126-7810), 1-200 μA filtered pipette tips (USA Scientific 1120-8810), sterile transfer pipettes (VWR 14670-147), 55 ml sterile solution basin (VWR 21007-974), 2 waste containers (one for liquid, one for tips/pipettes), 1.5 ml sterile microcentrifuge tubes (VWR 20170-038), Microcentrifuge tube rack, permanent marker, Eppendorf Microcentrifuge, model #5417C.

### Procedure:

1. Loosen the pellet in the microcentrifuge tubes by thawing slightly and then flick the tube to dislodge the pellet.

2. Add the appropriate volume of Buffer RLT (in this case use 600 μl). Vortex or pipette to mix.

3. Transfer sample to a QIAshredder tube to homogenize the sample. Centrifuge for 2 minutes at 14,000 rpm. Discard spin column but keep the collection tube and its contents.

4. Add one volume (600 μl) of 70% ethanol to the homogenized lysate and mix by pipetting.

5. Apply a 600 μl aliquot of the sample to an RNeasy mini column placed in a 2 ml collection tube. Close tube gently and centrifuge for 15 sec at 14,000 rpm. Discard the flow-through. Add the second 600 μl aliquot of the cell lysate to the same spin column and repeat. Discard flow-through.

6. Reuse the collection tube from step 5. Add 700 μl Buffer RW1 to the column. Centrifuge for 15 sec at 14,000 rpm. Discard the flow-through and collection tube.

7. Transfer the column to a new 2 ml collection tube and pipette 500 μl Buffer RPE onto the column. Centrifuge for 15 sec at 14,000 rpm to wash the column. Discard the flow-through but save the collection tube for step 8.

8. Add another 500 ml Buffer RPE to the column. Centrifuge for 2 min at 14,000 rpm to dry the membrane.

9. Transfer the column to a new 1.5 ml collection tube. Pipette 10 μl of RNA Storage Solution directly onto the membrane. Centrifuge for 1 min at 14,000 rpm to elute the RNA. Add a second volume of 5 μl of RNA Storage Solution directly to the membrane and spin for an additional minute. Store the final elution of RNA at −80° C.

### RNA Probe Preparation and Hybridization.

### Reagent:

Ovation TM Biotin System v1.0 for probe preps.

### Protocol:

User Guide (Cat#D01002, version Oct. 27, 2004, NuGEN Technologies, Inc). The experimental procedure is followed as described in the user guide. All probe preparation starts with 50 ng of total RNA.

### Gene Chip Procedures:

The Genechips used for the test is the Canine Genome 2.0 Array (Affymetrix). This Genechip contains 44,000 probe sets. Detailed sequence information for each unique probe identification number is available from the manufacturer.

### Gene Expression Analysis:

Normalization is performed using MAS 5 provided in GCOS Affymetrix software (version 1.2). Expression levels for the genes analyzed are indicated on the tables included in the examples below, where an upward facing arrow refers to “up regulation” or increase and a downward facing arrow indicates “down regulation” in gene expression. Similarly, in some tables, upward or downward facing arrows also indicate increases or decreases in activity of certain proteins involved in a particular pathway, and are otherwise self explanatory.

### Gene List Selection:

15,411 genes are chosen for further analysis based on their “present” calls in at least 9 out of 18 samples.

Results of the gene chip analysis indicate that 1088 genes are differentially expressed between the control and Super Senior diet treated groups. The expression levels of these 1088 genes are statistically significant when grouped by ‘diet’; using a parametric test where the variances is not assumed to be equal (Welch t-test). The p-value cutoff is 0.01 with no multiple testing correction. Under those selection criteria only about 154 genes would be expected to pass the restriction by chance. The genomic data is discussed in detail below.

### Results:

Effect of Nutrition on Genes Associated with Pain and Inflammation

Based on an analysis of the gene chip data, at the P<0.01 level, expression levels of 1,088 genes changed compared to control expression levels (10 were up regulated and the rest down regulated). At the P<0.001 level, data indicate that expression in 35 genes is down regulated in beagles fed the super senior food. Nine of these down regulated genes are identified as related to the inflammatory and pain response. Down regulation of these genes may be predicted to result in pain relief, cartilage protection (less damage) and reduction in inflammatory responses. The compositions disclosed herein may be part of a therapeutic regimen to treat animals suffering from pain and/or inflammatory diseases. These genes and their putative role in inflammation and pain response are provided below in Tables 5-6.

TABLE 5
Genes involved in inflammation and pain response (P < 0.001)

| | | | | | |
|---|---|---|---|---|---|
| | | | | % match of | |
| | | | | probe | |
| | | | Best Current BLAST | sequence to | Probe Target |
| Genes | Also Known As | Probe | Annotation | BLAST hit | Sequence |

| | | | | | |
|---|---|---|---|---|---|
| Phospholipase | IPLA2GAMMA, | CfaAffx.6431. | PREDICTED: Canis | 100 | GGAGCCATGCATTT |
| A2 | IPLA2-2 | 1.S1_s_at | familiaris similar to | | TATGACAGTCAAAC |
| | | | intracellular | | GTGGGAAAATATTC |
| | | | membrane-associated | | TTAAGGACAGAATG |
| | | | calcium-independent | | GGATCCTCGCTAAT |
| | | | phospholipase A2 | | GATTGAAACAGCAA |
| | | | gamma; transcript | | GAAACCCTTCATGT |
| | | | variant 3 | | CCTAAGGATGGAG |
| | | | (LOC475880); mRNA | | GTTTGCTTCTGAAT |
| | | | | | AACCCTTCAGCGCT |
| | | | | | AGCAATGCACGAGT |
| | | | | | GCAAATGTCTTTGG |
| | | | | | CCTGACGTCCCATT |
| | | | | | AGAGTGCATTGTGT |
| | | | | | CCCTGGGCACCGG |
| | | | | | GCGTTATGAGAGTG |
| | | | | | ATGTGAGAAACTCT |
| | | | | | GTGACATCTACAAG |
| | | | | | CTTGAAAACCAAAC |
| | | | | | TGTCTAATGTCATT |
| | | | | | AACAGTGCTACAGA |
| | | | | | TACAGAAGAAGTCC |
| | | | | | ACGTAATGCTTGAT |
| | | | | | GGTCTTTTACCTCC |
| | | | | | TGACACCTATTTTA |
| | | | | | GAT |
| Dipeptidase 2 | Putative | CfaAffx.31124. | PREDICTED: Canis | 82.197 | GTGCTGCAATGCAA |
| | dipeptidase | 1.S1_at | familiaris similar to | | CCTGTTAGCTAACG |
| | | | dipeptidase 2 | | TGTCCACTGTGGCA |
| | | | (LOC611083); mRNA | | GTTCCCACGCATCC |
| | | | | | CTGCCCTGGAAGC |
| | | | | | CCCACAGTGCTGAC |
| | | | | | TCTCCATCCCTCAG |
| | | | | | ATCACTTTGACTAC |
| | | | | | ATCAGGGCAGTCAT |
| | | | | | TGGATCCAAGTTCA |
| | | | | | TTGGAATTGGTGGA |
| | | | | | GATTATGATGGGGC |
| | | | | | CAGACGTTTCCCTC |
| | | | | | AGGGGCTGGAGGA |
| | | | | | TGTGTCCACATACC |
| | | | | | CAGTTCTGATAGAG |
| | | | | | GAGTTGCTGAGGC |
| | | | | | GTGGCTGGAGTAG |
| | | | | | GGAAGAGCTCCAG |
| | | | | | GGTGTCCTTCGAG |
| | | | | | GAAACCTACTGCGG |
| | | | | | GTCTTTGGACAGGT |
| | | | | | GGAACAGGTACGG |
| | | | | | GAGGCAAGCAAGG |
| | | | | | GGCAAAGGCCCTT |
| | | | | | GGAGGATGAGTTC |
| | | | | | CCGGATGAGCAGC |
| | | | | | TGAGCAGCTCTTGC |
| | | | | | CGCTCCGTTCTCTC |
| | | | | | ACGTCTGCATCAGA |
| | | | | | CACAGTACCCTGCT |
| | | | | | CCATACCAGAAACT |
| | | | | | AACTGAGATTTCAC |
| | | | | | CTGAGTGGTCCCCT |
| | | | | | AAACAGTCATTGTC |
| | | | | | AAAATCTCTCCCCA |
| | | | | | TCATGGCCCCAGG |
| | | | | | CCTCATAGTTATTG |
| | | | | | CTGCTTGT |
| Thromboxane | Thromboxane A | CfaAffx.6939. | PREDICTED: Canis | 100 | ATCGCTGGCTATGA |
| synthase | synthase 1, | 1.S1_s_at | familiaris similar to | | GATCATCACCAACA |
| | Thromboxane A | | Thromboxane-A | | CGCTCTCTTTTGCC |
| | synthase, Platelet, | | synthase (TXA | | ACCTACCTCCTGGC |
| | Cytochrome P450, | | synthase) (TXS) | | CACCAACCCTGACT |
| | subfamily V, | | (LOC482771); mRNA | | GCCAAGAGAAGCTT |
| | CYP5, CYP5A1, | | | | CTGGCAGAGGTGG |
| | Thromboxane | | | | ACAGCTTTAAGGAG |
| | synthatase, TXA | | | | AAATATACGGCCCT |
| | synthase, TXS | | | | TGACTACTGCAGCC |
| | | | | | TCCAGGAAGGCCT |
| | | | | | GCCCTACCTGGACA |
| | | | | | TGGTGATTGCGGA |
| | | | | | GACCTTGAGGATCT |
| | | | | | ACCCCCCGGCTTTC |
| | | | | | AGGTTCACACGGG |
| | | | | | AGGCGGCGCGGGA |
| | | | | | CTGCGAGGTGCGG |
| | | | | | GGACAGCGCATCC |
| | | | | | CCGCGGGCGCCGT |
| | | | | | GGTGGAGGTGGCC |
| | | | | | GTGGGCGCCCTGC |
| | | | | | ACCGTGACCCTGA |
| | | | | | GTACTGGCCACAAC |
| | | | | | CGGAGACCTTCAAC |
| | | | | | CCCGAGAGGTTCAA |
| | | | | | GGCCGAGGCGCAG |
| | | | | | CGACGACAGCAAC |
| | | | | | CCTTCACCTACCTG |
| | | | | | CCGTTCGGCGCGG |
| | | | | | GCCCCCGGAGCTG |
| | | | | | CCTCGGGGTGCGG |
| | | | | | CTGGGGCTGCTGG |
| | | | | | AGGTCAAGCTGAC |
| | | | | | GCTGCTGCAGGTC |
| | | | | | CTGCACCAGTTCCG |
| | | | | | GTTCGAGGCCTGC |
| | | | | | CCGGAGACGCAGG |
| | | | | | TACCACTGCAGCTA |
| | | | | | GACTCCAAATCTGC |
| | | | | | CCTAGGTCCAAAGA |
| | | | | | ATGGCATCTACATC |
| | | | | | AAGATTGTCTCCCG |
| | | | | | CT |
| Ubiquitin | Ubiquitin protein | CfaAffx.275.1. | PREDICTED: Pan | 97.19626 | GATTTGGCCCGTGA |
| conjugating | ligase, Ubiquitin | S1_s_at | troglodytes | | CCCTCCAGCACAAT |
| enzyme | carrier protein, | | LOC461941 | | GTTCTGCAGGTCCT |
| E2D 3 | E2(17)KB 3, | | (LOC461941); mRNA | | GTTTGGGATGATAT |
| | Ubiquitin | | | | GTTTCATTGGCAAG |
| | conjugating | | | | CCACAATTATAGGA |
| | enzyme E2-17 kDa | | | | CCTAATGACAGCCC |
| | 3, UBC4/5, | | | | ATATCAAGG |
| | UBCH5C | | | | |
| NEDD8 | Neural precursor | Cfa.12556.1.A | PREDICTED: Canis | 99.12473 | GGAATGGGCTACTC |
| ultimate | cell expressed, | 1_s_at | familiaris similar to | | TACTCATGCAGNCA |
| buster-1 | developmentally | | NEDD8 ultimate | | AGCAGGNCCTGCA |
| | down regulated 8, | | buster-1 (NY-REN-18 | | TCAGGCCAGTGGG |
| | Ubiquitin like | | antigen) | | AACCTGGACGAAG |
| | protein NEDD8 | | (LOC475542); mRNA | | CCCTGAAGATTCTT |
| | | | | | CTCAGCAATCCTCA |
| | | | | | GATGTGGTGGTTAA |
| | | | | | ATGATTCAGATCCT |
| | | | | | GAAACGANCAACCA |
| | | | | | GCAAGAAAGTCCTT |
| | | | | | CCCAGGAAAACATT |
| | | | | | GACCAACTGGTGTA |
| | | | | | CATGGGCTTCGAC |
| | | | | | GCTGTGGTGGCTG |
| | | | | | ATGCTGCCTTGAGA |
| | | | | | GTGTTCAGGGGAAA |
| | | | | | CGTGCAGCTGGCA |
| | | | | | GCTCAGNCCCTCG |
| | | | | | CCCACAACGGAGG |
| | | | | | AACTCTTCCTCCTG |
| | | | | | ACCTGCAGCTCTTG |
| | | | | | GTGGAAGACTCTTC |
| | | | | | ATCAACGCCATCCA |
| | | | | | CGTCCCCTTCCGAC |
| | | | | | TCCGCAGGTACCTC |
| | | | | | TAGTGCCTCAACAG |
| | | | | | ATGAAGATATGGAA |
| | | | | | ACCGAAGCTGTCAA |
| | | | | | TGAAATACTGGAAG |
| | | | | | ATATTCCAGAACAT |
| | | | | | GAAGAAGATTATCT |
| | | | | | TGACTCAACACTGG |
| | | | | | AAG |
| Mitogen- | p38, Mitogen | CfaAffx.2947. | Homo sapiens | 97.84946 | GAGATGGAGTCCT |
| activated | activated protein | 1.S1_at | mitogen-activated | | GAGCACCTGGTTTC |
| protein | kinase 14, | | protein kinase 14; | | TGTTTTGTTGATCC |
| kinase 14 | Cytokine | | transcript variant 2; | | CACTTCACTGTGAG |
| (p38) | suppressive | | mRNA (cDNA clone | | GGGAAGGCCTTTTC |
| | antiinflammatory | | MGC: 34610 | | ATGGGAACTCTCCA |
| | drug binding | | IMAGE: 5181064); | | AATATCATTC |
| | protein 1, CSBP1, | | complete cds | | |
| | CSAID binding | | | | |
| | protein 1, Stress | | | | |
| | activated protein | | | | |
| | kinase 2A, | | | | |
| | SAPK2A, p38 | | | | |
| | MAP kinase, p38 | | | | |
| | alpha, RK, MXI2, | | | | |
| | Cytokine | | | | |
| | suppressive | | | | |
| | antiinflammatory | | | | |
| | drug binding | | | | |
| | protein 2, CSBP2, | | | | |
| | CSAID binding | | | | |
| | protein 2 | | | | |
| Matrix | MMP 19 | Cfa.4573.1.A1_at | Homo sapiens cDNA | 48.93048 | GTAGTTGATTCCTG |
| metalloproteinase | | | FLJ38021 fis; clone | | GTTCGCCTTTCCTC |
| 19 | | | CTONG2012847 | | TTGGGTCCCATAGG |
| (MMP-19) | | | | | TTCGAATCCCCTTC |
| | | | | | TACCTCAGTCGGGA |
| | | | | | GTACTGTCCTCCAT |
| | | | | | GGTGCTTCCCTTCC |
| | | | | | TCTCCTTAATGTGG |
| | | | | | GGAAGACCATGGG |
| | | | | | GCAATGCATGGCG |
| | | | | | CAGGACCTGCCTC |
| | | | | | CCCCAAAAGCAGTC |
| | | | | | TACTTGCTCCACGG |
| | | | | | AGAGAGAACTGGG |
| | | | | | TCCACGTGCCAGA |
| | | | | | GTCTTGCCCTTTGG |
| | | | | | CCCAGAGTAGCCT |
| | | | | | GGTCTTCATGGCTG |
| | | | | | TATGGGAGACAAGT |
| | | | | | GCCTTCTCTGCTTC |
| | | | | | TTGTTGTAGGTGAT |
| | | | | | GCTAATCTCCTTAA |
| | | | | | CCAAACCTTTGTCC |
| | | | | | CAGCCGCTAATCTG |
| | | | | | TTCTAACTCTCCCT |
| | | | | | CCTCNTGATTCTCC |
| | | | | | TGCTCAAAGTCTGT |
| | | | | | TC |
| Tissue | TIMP-1 | Cfa.3680.1.S1_s_at | Canis familiaris TIMP | 99.4 | AGATGTTCAAGGGT |
| Inhibitor of | | | metallopeptidase | | TTCAGCGCCTTGGG |
| metalloproteinases | | | inhibitor 1 (TIMP1); | | GAATGCCTCGGACA |
| (TIMP-1) | | | mRNA | | TCCGCTTCGTCGAC |
| | | | | | ACCCCCGCCCTGG |
| | | | | | AGAGCGTCTGCGG |
| | | | | | ATACTTGCACAGGT |
| | | | | | CCCAGAACCGCAG |
| | | | | | CGAGGAGTTTCTGG |
| | | | | | TCGCCGGAAACCT |
| | | | | | GCGGGACGGACAC |
| | | | | | TTGCAGATCAACAC |
| | | | | | CTGCAGTTTCGTGG |
| | | | | | CCCCGTGGAGCAG |
| | | | | | CCTGAGTACCGCTC |
| | | | | | AGCGCCGGGGCTT |
| | | | | | CACCAAGACCTATG |
| | | | | | CTGCTGGCTGTGA |
| | | | | | GGGGTGCACAGTG |
| | | | | | TTTACCTGTTCATC |
| | | | | | CATCCCCTGCAAAC |
| | | | | | TGCAGAGTGACACT |
| | | | | | CACTGCTTGTGGAC |
| | | | | | GGACCAGTTCCTCA |
| | | | | | CAGGCTCTGACAAG |
| | | | | | GGTTTCCAGAGCC |
| | | | | | GCCACCTGGCCTG |
| | | | | | CCTGCCAAGAGAG |
| | | | | | CCAGGGATATGCAC |
| | | | | | CTGGCAGTCCCTG |
| | | | | | CGGCCCCGGATGG |
| | | | | | CCTAAATCCTACTC |
| | | | | | CCCGTGGAAGCCA |
| | | | | | AAGCCTGCACAGTG |
| | | | | | TTCACCCCACTTCC |
| | | | | | CACTCCTGTCTTTC |
| | | | | | TTTATCCAAAA |
| Fatty acid | Oleamide | CfaAffx.7308. | PREDICTED: Canis | 63.33333 | GAAGTGGAGTAGG |
| amide | hydrolase | 1.S1_x_at | familiaris similar to | | TGCCGCTGTTGCTG |
| hydrolase | Anandamide | | Ubiquinol-cytochrome | | CTGGTGTTGAATTC |
| (FAAH) | amidohydrolase | | c reductase complex | | AGAACTGTAGCGG |
| | FAAH | | 11 kDa protein; | | GACATGGGGCTGG |
| | | | mitochondrial | | AGGACGAGCAAAA |
| | | | precursor | | GATGCTGACCGGG |
| | | | (Mitochondrial hinge | | TCCGGAGATCCCAA |
| | | | protein) (Cytochrome | | GGAGGATCCCCTAA |
| | | | C1; nonheme 11 kDa | | CAACAGTGAGAGA |
| | | | protein) (Complex III | | GCAATGCGAGCAG |
| | | | subunit VIII); | | CTGGAGAAATGTGT |
| | | | transcript variant 2 | | AAAGGCTCGGGAG |
| | | | (LOC608530); mRNA | | CGGCTAGAGCTCT |
| | | | | | GTGACCAGCGTGTA |
| | | | | | TCCTCCAGGTCACA |
| | | | | | GACAGAGGAGGAT |
| | | | | | TGCACAGAGGAGC |
| | | | | | TCTTTGACTTCCTG |
| | | | | | CATGCAAGGGACC |
| | | | | | ACTGTGTGGCCCAC |
| | | | | | AAACTCTTTAACAG |
| | | | | | CTTG |

TABLE 6
Summary of down-regulated enzyme roles involved
in the eicosanoid pathway (inflammatory response)

| | Gene Expression | | |
|---|---|---|---|
| | Compared to | | |
| Gene | Control | Results in | Role |
| Phospholipase | ↓ | ↓ in arachidonic | ↓ in 2-series inflammatory |
| A₂ | | release from | response |
| | | phospholipids | |
| Thromboxane | ↓ | ↓ Thromboxane A₂ | ↓ platelet aggregation, |
| synthase | | | vasoconstriction, lymphocyte |
| | | | proliferation and |
| | | | bronchoconstriction |
| | ↓ | ↓ Thromboxane B₂ | ↓ vasoconstriction |
| Dipeptidase 2 | ↓ | ↓ Leukotriene E₄ | ↓ component of slow-reactive |
| | | | substance of anaphylaxis, |
| | | | microvascular vasoconstrictor |
| | | | and bronchoconstriction |
| Ubiquitin | ↓ | ↓ ubiquination or | ↓ MMP Production |
| conjugating | | activation of TAK1, | |
| enzyme E2D 3 | | IRAK and TRAF | |
| (and NEDD8 | | | |
| ultimate | | | |
| buster-1) | | | |
| Mitogen | ↓ | ↓ in c-Jun promotor | ↓ MMP Production |
| activated | | | |
| protein kinase | | | |
| 14 (p38) | | | |
| MMP-19 | ↓ | ↓ MMP-19 | ↓ in T-cell derived MMP-19 |
| | | | which has been implicated in |
| | | | rheumatoid arthritis |
| TIMP-1 | ↓ | ↓ TIMP-1 | Deactivates MMP's |
| | | | concentration is directly |
| | | | related to MMP concentration |
| Fatty acid | ↓ | ↑ anandmide | ↓ pain response |
| amide | | | |
| hydrolase | | | |

### Effect of Nutrition on Genes Involved in Heart Health and Blood Coagulation

At the P<0.001 and P<0.01 level, 12 genes are identified to be related to heart health through regulation of the eicosanoid pathway and blood coagulation pathway. The genes are responsible for blood coagulation through platelet activation and aggregation. The down regulation of these genes through nutrition can prevent inappropriate blood clotting which may result in heart or brain related disorders. The compositions of the present invention may be part of a therapeutic regimen to treat animals suffering from disorders or diseases of the blood, heart or brain. These genes and their putative role in vivo are described in Tables 7 and 8 below.

TABLE 7
Genes involved in heart health and blood coagulation

| | | | | | |
|---|---|---|---|---|---|
| | | | | % match of | |
| | | | Best current | probe | |
| | | | BLAST | sequence to | |
| Gene | Probe | P-value | annotation | BLAST hit | Probe Target Seq. |

| | | | | | |
|---|---|---|---|---|---|
| Glycoprotein | Cfa.3503.1. | <0.01 | Canis familiaris | 98.57143 | TGTGGGTCCGAGCTAACAGCTA |
| Ib | S1_at | | glycoprotein lb | | CGTGGGGCCTCTGATGGCAGG |
| | | | mRNA; complete | | ACGGCGGCCCTCTGCCCTGAG |
| | | | cds | | CCTGGGTCGTGGGCAGGACCT |
| | | | | | GCTAGGTACGGTGGGCGTTAG |
| | | | | | GTACTCCAGCCACAGCCTCTGA |
| | | | | | GGCGACGGTGGGCAGTTTGGG |
| | | | | | GACCTTGAGAGGCTGTGATGG |
| | | | | | GCCCTCCTATCAGGATCTTGCT |
| | | | | | GGGGGTGGGTGGGCAGGGAG |
| | | | | | CACAGGATTGGGGGGAGGCCT |
| | | | | | TAAGCACCTTTTCTGGGTCAGA |
| | | | | | AGCCTCCTCTCCGCATTGCATG |
| | | | | | TGCAACCTCAGTGAAGCAGCAT |
| | | | | | GGGCAGGGGAGCCGGACGGG |
| | | | | | CCACCCAACAGAGCTCCTTATG |
| | | | | | CTGCAGGAGGGGTTCACAGAC |
| | | | | | CACTCGGACATCACCATCACCT |
| | | | | | TGGGGGGGGTGCTTGAGGGAA |
| | | | | | AAGCAAATTGAACAGAGCGTGA |
| | | | | | TTCTCACGTGCAGGTACCTAAG |
| | | | | | GGAACTGGGGAAGAGATGCAC |
| | | | | | CAAGACGAGAGCCCTCGTCATC |
| | | | | | CCTGGGGAGCCCAAGCCTAGG |
| | | | | | GGTTTTCTTCCTCTTCCCGTTTA |
| | | | | | GCATTTTCCACCATCGTATGTTAC |
| Platelet | CfaAffx.4809. | <0.01 | PREDICTED: | 50 | AGTTTTGACCAATTCGCTCTGT |
| glycoprotein | 1.S1_at | | Canis familiaris | | ACAAGGAGGGGGACACTGAGC |
| VI | | | similar to | | CCCACAAGCAATCTGCAGAACA |
| | | | glycoprotein VI | | GTACTGGGCCAATTTCCCCATC |
| | | | (platelet) | | ACCGCAGTGACTGTTGCCCACA |
| | | | (LOC484303); | | GTGGGATCTACCGATGCTATAG |
| | | | mRNA | | CTTTTCCAGCAAGTTCCCGTAC |
| | | | | | CTGTGGTCAGCCCCCAGCGAC |
| | | | | | CCCCTGGAGCTTGTGGTAACAG |
| | | | | | GTGAGGGAGATGCAGTCCAAG |
| | | | | | CCTTTCTTCTTCAGCTCTTGCAT |
| | | | | | ACTCTGGTGGAAGTTCCAGGG |
| | | | | | GAGGGGCCAACAGTGCCTTCT |
| | | | | | AGGACTATCACTGTCTCTCCAA |
| | | | | | AGGGGTCAGACTCTCCAACTG |
| | | | | | GTCTTGCTCACCAGCACTACAC |
| | | | | | CAAGGGCAATCTGGTCCGGATA |
| | | | | | TGCCTTGGAGCTGTGATTCTAA |
| | | | | | TACTCCTGGTGGGAATTCTGGC |
| | | | | | AGAAGATTGGCACAGCAGAAAG |
| | | | | | AAACCCCTGTTGCTCCGGGTCA |
| | | | | | GAGCTGTCCACAGGCCACTCC |
| | | | | | CACCCCTCCCACAGACCCAGAA |
| | | | | | ACCACACAGTCATCAGGATGGG |
| | | | | | GGTCGACCAGATGGCCATAAC |
| | | | | | CAT |
| Platelet | CfaAffx.7430. | <0.01 | PREDICTED: | 100 | TCTGGGCTGCCACGGAGGCCA |
| glycoprotein | 1.S1_at | | Canis familiaris | | CCAACGACTGCCCCGCAGAGT |
| IX precursor | | | similar to Platelet | | GCACCTGCCAGACCCTGGAGA |
| | | | glycoprotein IX | | CCATGGGGCTGTGGGTGGACT |
| | | | precursor (GPIX) | | GCAGGGGGCGGGGACTCAAGG |
| | | | (CD42A) | | CCCTGCCCGCCCTGCCGGTCC |
| | | | (LOC609630); | | ACACCCGCCACCTCCTGCTGG |
| | | | mRNA | | CCAATAACAGCCTCCGCTCCGT |
| | | | | | GCCCCCTGGTGCCTTCGACCA |
| | | | | | CCTGCCTGGGCTGCAGATCCT |
| | | | | | CGACGTGATGCACAACCCCTG |
| | | | | | GCACTGTGACTGCAGCCTCACC |
| | | | | | TACCTGCGTCTCTGGCTGGAG |
| | | | | | GACCACACGCCCGAGGCCTTG |
| | | | | | CTGCAGGTCCGCTGTGCCAGC |
| | | | | | CCCGCGCTGGCCACCACCCGG |
| | | | | | CCGCTGGGCTGGCTGACGGGC |
| | | | | | TACGAGCTGGGCAGCTGCGGC |
| | | | | | TGGCAGCTACAGGCACCCTGG |
| | | | | | ACCTA |
| Coagulation | CfaAffx.14964. | <0.01 | PREDICTED: | 99.6008 | ATCTCTCAGGCAACATCGTCTT |
| factor XIII A | 1.S1_s_at | | Canis familiaris | | CTACACCGGGGTCTCCAAGAC |
| chain | | | similar to | | GGAATTCAAGAAGGAGACATTT |
| precursor | | | Coagulation | | GAAGTGACACTGGAGCCCTTGT |
| | | | factor XIII A | | CTTTCAAGAGAGAGGAGGTGCT |
| | | | chain precursor | | GATCAGAGCGGGCGAGTACAT |
| | | | (Coagulation | | GGGCCAGCTGCTAGAGCAAGC |
| | | | factor XIIIa) | | ATACCTGCACTTCTTTGTCACA |
| | | | (Protein- | | GCGCGTGTCAATGAGTCCAAG |
| | | | glutamine | | GATATTCTGGCCAAGCAGAAGT |
| | | | gamma- | | CCACCGTGCTGACGATCCCCC |
| | | | glutamyltransferase | | AGCTCATCATCAAGGTCCGTGG |
| | | | A chain) | | CGCCAAGATGGTTGGTTCTGAC |
| | | | (Transglutaminase | | ATGGTGGTGACAGTTGAGTTCA |
| | | | A chain); | | CCAATCCCCTGAAAGAAACTCT |
| | | | transcript variant | | GCGGAATGTGTGGATACACCTG |
| | | | 1 (LOC478711); | | GATGGTCCTGGAGTGATAAAGC |
| | | | mRNA | | CAATGAGGAAGATGTTCCGTGA |
| | | | | | AATCCAGCCCANTGCCACCATA |
| | | | | | CAATGGGAAGAAGTGTGTCGAC |
| | | | | | CCTGGGTGTCTGGCCCTCGGA |
| | | | | | AGCTGATAGCCAGCATGACGA |
| | | | | | GTGACTCCCTGAGACACGTGTA |
| | | | | | TG |
| Thromboxane | CfaAffx.6939. | <0.001 | PREDICTED: | 100 | ATCGCTGGCTATGAGATCATCA |
| synthase | 1.S1_s_at | | Canis familiaris | | CCAACACGCTCTCTTTTGCCAC |
| | | | similar to | | CTACCTCCTGGCCACCAACCCT |
| | | | Thromboxane-A | | GACTGCCAAGAGAAGCTTCTGG |
| | | | synthase (TXA | | CAGAGGTGGACAGCTTTAAGGA |
| | | | synthase) (TXS) | | GAAATATACGGCCCTTGACTAC |
| | | | (LOC482771); | | TGCAGCCTCCAGGAAGGCCTG |
| | | | mRNA | | CCCTACCTGGACATGGTGATTG |
| | | | | | CGGAGACCTTGAGGATCTACCC |
| | | | | | CCCGGCTTTCAGGTTCACACGG |
| | | | | | GAGGCGGCGCGGGACTGCGA |
| | | | | | GGTGCGGGGACAGCGCATCCC |
| | | | | | CGCGGGCGCCGTGGTGGAGGT |
| | | | | | GGCCGTGGGCGCCCTGCACCG |
| | | | | | TGACCCTGAGTACTGGCCACAA |
| | | | | | CCGGAGACCTTCAACCCCGAG |
| | | | | | AGGTTCAAGGCCGAGGCGCAG |
| | | | | | CGACGACAGCAACCCTTCACCT |
| | | | | | ACCTGCCGTTCGGCGCGGGCC |
| | | | | | CCCGGAGCTGCCTCGGGGTGC |
| | | | | | GGCTGGGGCTGCTGGAGGTCA |
| | | | | | AGCTGACGCTGCTGCAGGTCC |
| | | | | | TGCACCAGTTCCGGTTCGAGG |
| | | | | | CCTGCCCGGAGACGCAGGTAC |
| | | | | | CACTGCAGCTAGACTCCAAATC |
| | | | | | TGCCCTAGGTCCAAAGAATGGC |
| | | | | | ATCTACATCAAGATTGTCTCCC |
| | | | | | GCT |
| Dystrobrevin | CfaAffx.15541. | <0.01 | PREDICTED: | 99.65986 | GGCAACATGTCGTCCATGGAG |
| binding | 1.S1_s_at | | Canis familiaris | | GTCAACATCGACATGCTGGAGC |
| protein 1 | | | similar to | | AGATGGACCTGATGGACATCTC |
| isoform a | | | dystrobrevin | | TGACCAGGAGGCCCTGGACGT |
| | | | binding protein 1 | | CTTCCTGAACTCCGGCGCTGAA |
| | | | isoform a | | GACAACACGGTGCCGTCTCCG |
| | | | (LOC610315); | | GTCTCAGGGCCTGGCTCGGGG |
| | | | mRNA | | GACAGTCGGCAGGAAATCACG |
| | | | | | CTCCGGGTTCCAGATCCCGCC |
| | | | | | GAATCGCAAGCTGAGCCTCCTC |
| | | | | | CCTCGCCGTGTGCCTGTCCTGA |
| | | | | | GCTGGCCGCCCCGGCCCCCGG |
| | | | | | CGACGGTGAGGCCCCCGTGGT |
| | | | | | CCAGTCTGACGAGGAG |
| Integrin beta-7 | Cfa.11961.1. | <0.01 | PREDICTED: | 99.0909 | ATTACAACGTGACTCTGGCTTT |
| precursor | A1_s_at | | Canis familiaris | | GGTCCCTGTCCTGGATGACGG |
| | | | similar to Integrin | | CTGGTGCAAAGAGAGGACCCT |
| | | | beta-7 precursor | | AGACNAACCAGCTGCTGTTCTT |
| | | | (LOC477598); | | CCTGGTGGAGGAGGAANCCGG |
| | | | mRNA | | AGGCATGGTTGTGTTGACAGTG |
| | | | | | AGACCCCAAGAGAGAGGCGCG |
| | | | | | GATCACACCCAGGCCATCGTG |
| | | | | | CTGGGCTGTGTAGGGGGCATC |
| | | | | | GTGGCAGTGGGGCTGGGGCTG |
| | | | | | GTCCTGGCTTACCGGCTCTCTG |
| | | | | | TGGAAATCTACGNCCGCCGAG |
| | | | | | AATTTAGCCGCTTTGAGAAGGA |
| | | | | | GCAGAAGCACCTCAACTGGAA |
| | | | | | GCAGGAAAACAATCCTCTCTAC |
| | | | | | AGAAGCGCC |
| integrin-linked | Cfa.465.1.S1_s_at | <0.01 | PREDICTED: | 100 | TGGGCGCATGTATGCACCTGC |
| kinase | | | Canis familiaris | | CTGGGTGGCCCCTGAAGCTCT |
| | | | similar to integrin | | GCAGAAGAAGCCTGAAGATACA |
| | | | linked kinase; | | AACAGACGCTCAGCAGATATGT |
| | | | transcript variant | | GGAGTTTTGCAGTGCTTCTGTG |
| | | | 1 (LOC476836); | | GGAACTGGTGACGAGGGAGGT |
| | | | mRNA | | ACCCTTTGCTGACCTCTCCAAC |
| | | | | | ATGGAGATTGGAATGAAGGTGG |
| | | | | | CACTGGAAGGCCTTCGGCCTA |
| | | | | | CTATCCCACCAGGCATTTCCCC |
| | | | | | CCATGTGTGTAAGCTCATGAAG |
| | | | | | ATCTGCATGAATGAAGACCCTG |
| | | | | | CTAAGCGGCCCAAGTTTGACAT |
| | | | | | GATTGTGCCTATCCTGGAGAAG |
| | | | | | ATGCAGGACAAGTAGAGCTGG |
| | | | | | AAAGCCCTTGCCTAAACTCCAG |
| | | | | | AGGTGTCAGGACACGGTTAGG |
| | | | | | GGAGTGTGTCTCCCCAAAGCA |
| | | | | | GCAGGC |
| Thrombospondin 1 | Cfa.21204.1. | <0.01 | PREDICTED: | 54.83871 | ATACGAATGCAGAGATTCCTAA |
| | S1_at | | Canis familiaris | | TCAAACTGTTGATCAAAAGACT |
| | | | similar to | | GATCCTAACCAATGCTGGTGTT |
| | | | thrombospondin | | GCACCTTCTGGAACCACGGGC |
| | | | 1 precursor | | TTAAGAAAACCCCCAGGATCAC |
| | | | (LOC487486); | | TCCTCCCTGCCTTTTCTCTGCTT |
| | | | mRNA | | GCATATCATTGTGGACACCTAG |
| | | | | | AATACGGGACTTGCCTCGAGAC |
| | | | | | CATGCNNNNNTCCAAATCAGAC |
| | | | | | TNNNNNNGTAGCCTCTGAACGC |
| | | | | | GAAGAGAATCTTCCAAGAGCAT |
| | | | | | GAACAG |
| Thrombospondin | CfaAffx.18675. | <0.01 | PREDICTED: | 100 | GAAGCCCTTGATGGATACTGTG |
| repeat | 1.S1_s_at | | Canis familiaris | | AACGGGAACAGGCTATAAAGAC |
| containing 1 | | | similar to | | CCACCACCACTCCTGTTGCCAC |
| | | | extracellular | | CACCCTCCTAGCCCTGCCCGC |
| | | | matrix protein 1 | | GATGAGTGCTTTGCCCGTCAGG |
| | | | isoform 1 | | CGCCATACCCCAACTATGACCG |
| | | | precursor | | GGACATCCTGACCCTTGATTTC |
| | | | (LOC608791); | | AGCCAAGTTACCCCCAACCTCA |
| | | | mRNA | | TGCAACATCTCTGTGGAAATGG |
| | | | | | AAGACTTCTCACCAAGCATAAA |
| | | | | | CAGATTCCTGGGCTGATCCGGA |
| | | | | | ACATGACTGCCCACTGCTGTGA |
| | | | | | CCTGCCATTTCCAGAGCAGGCC |
| | | | | | TGCTGTGCTGAGGAGGAGAAAT |
| | | | | | CGGCCTTCATTGCAGACTTGTG |
| | | | | | TGGTTCCCGACGTAACTTCTGG |
| | | | | | CGAGACTCTGCCCTCTGCTGTA |
| | | | | | ACCTGAATCCTGGAGATGAACA |
| | | | | | GACCAACTGCTTCAACACTTAT |
| | | | | | TATCTGAGGAATGTGGCTCTAG |
| | | | | | TGGCTGGAGACAAT |
| Thrombospondin | CfaAffx.16694. | <0.01 | PREDICTED: | 98.13084 | TGGTTGTAGCTCCTCACTTGTC |
| type 1 | 1.S1_at | | Canis familiaris | | CAAGACCGAAGCAGCAACCAAA |
| motif, 17 | | | similar to lines | | CTGAACTTAGCCTTTGGGCTGC |
| | | | homolog 1 | | TCTTGGTAGTCACAGAAATGCC |
| | | | isoform 1 | | CACGCTTCAGTCCCCTGGGCTT |
| | | | (LOC607902); | | CCAATGCTTCTGGACCTCTGAA |
| | | | mRNA | | CCAGCCTGTGATGTCCAAGGAA |
| | | | | | CCCCACGTCACGCTCCAGGCT |
| | | | | | GCTGCTGGTCTGTCTCCCCCAC |
| | | | | | AAGCTTCTCAAAGTCTGGTAGA |
| | | | | | TTATGACAGCTCTGATGATTCT |
| | | | | | GAAGTAGAAGTCACAGACCAGC |
| | | | | | ACTCAACAAACAGTAAACAAAC |
| | | | | | ATCTTTACAGCAAGAAGCAAAG |
| | | | | | AAGAAATTTCAGGACACAGTTA |
| | | | | | GAACAGGTCCAGATGAAAAAGA |
| | | | | | ACTTAGCATGGAGCCTCAATCA |
| | | | | | AGGCCTCTGGTTCCAGAACAAT |
| | | | | | CTAATATTAATATTCCCTTCTCT |
| | | | | | GTTGACTGTGACATCTCCAAAG |
| | | | | | TAGGAATATCTTACAGGACACT |
| | | | | | GAAGTGCTTTCAGGAGCTACAG |
| | | | | | GGTGCCATTTACCGTTTGCAGA |
| | | | | | AAAAAAATCTTTTCCCCTATAAT |
| | | | | | GCCACA |
| Angio- | Cfa.8616.1. | <0.001 | Canis familiaris | 64.77273 | GCGGACTGTGTTCCAACCCCTT |
| associated | A1_s_at | | angio-associated | | CAGCCGACTTGCCCCCTCCGT |
| migratory cell | | | migratory cell | | CCCTTCTCTTAAGAGACCCATC |
| protein | | | protein (AAMP) | | CCTTGGCCCCCCACCCCACCC |
| (AAMP) | | | gene; complete | | TCACCCAGACCTGCGGGTCCC |
| | | | cds | | TCAGAGGGGGGTCAGGCCTCT |
| | | | | | TTCTCTTTCACCTTCATTTGCTG |
| | | | | | GCGTGAGCTGCGGGGGTGTGT |
| | | | | | GTTTGTATGTGGGGAGTAGGTG |
| | | | | | TTTGAGGTTCCCGTTCTTTCCC |
| | | | | | TTCCCAAGTCTCTGGGGGTGGA |
| | | | | | AAGGAGGAAGAGATATTAGTTA |
| | | | | | CAGA |

TABLE 8
Summary of down regulated enzyme roles
involved in heart health and blood coagulation

| | Gene | |
|---|---|---|
| | Expression | |
| | compared | |
| Gene | to Control | Role |
| Glycoprotein Ib | ↓ | GP-Ib, a surface membrane |
| | | protein of platelets, |
| | | participates in the formation |
| | | of platelet plugs by binding |
| | | to the A1 domain of von |
| | | Willebrand factor, which is |
| | | already bound to the |
| | | subendothelium. |
| Platelet glycoprotein VI | ↓ | Collagen receptor belonging |
| | | to the immunoglobulin-like |
| | | protein family that is |
| | | essential for platelet |
| | | interactions with collagen |
| Platelet glycoprotein IX | ↓ | The GPIb-V-IX complex |
| precursor | | functions as the von |
| | | Willebrand factor receptor |
| | | and mediates von |
| | | Willebrand factor- |
| | | dependent platelet adhesion |
| | | to blood vessels. The |
| | | adhesion of platelets to |
| | | injured vascular surfaces in |
| | | the arterial circulation is a |
| | | critical initiating event in |
| | | hemostasis |
| Coagulation factor XIII A | ↓ | Factor XIII is activated by |
| chain precursor | | thrombin and calcium ion to |
| | | a transglutaminase that |
| | | catalyzes |
| | | the formation of gamma- |
| | | glutamyl-epsilon-lysine |
| | | cross-links between fibrin |
| | | chains, thus |
| | | stabilizing the fibrin clot. |
| Thromboxane synthase | ↓ | ↓ platelet aggregation, |
| | | vasoconstriction, |
| | | lymphocyte proliferation |
| | | and bronchoconstriction |
| Angio-associated | ↓ | contains a heparin-binding |
| migratory cell protein | | domain (dissociation |
| (AAMP) | | constant, 14 pmol) and |
| | | mediates heparin-sensitive |
| | | cell adhesion |
| Dystrobrevin binding | ↓ | Plays a role in the |
| protein 1 isoform a | | biogenesis of lysosome- |
| | | related organelles such as |
| | | platelet dense |
| | | granule and melanosomes |
| Thrombospondin 1 | ↓ | Adhesive glycoprotein that |
| | | mediates cell-to-cell and |
| | | cell-to-matrix interactions. |
| | | Can bind to fibrinogen, |
| | | fibronectin, laminin, type V |
| | | collagen and integrins |
| | | alpha-V/beta-1, alpha- |
| | | V/beta-3 and alpha-IIb/beta- |
| | | 3. |
| Thrombospondin type 1 | ↓ | Metalloprotease activity |
| motif, 17 | | |
| Thrombospondin repeat | ↓ | |
| containing 1 | | |
| Integrin beta-7 precursor | ↓ | Integrin alpha-4/beta-7 |
| | | (Peyer's patches-specific |
| | | homing receptor LPAM-1) |
| | | is expected to play a role in |
| | | adhesive interactions of |
| | | leukocytes. It is a receptor |
| | | for fibronectin and |
| | | recognizes one or more |
| | | domains within the |
| | | alternatively spliced CS-1 |
| | | region of fibronectin. |
| | | Integrin alpha-4/beta-7 is |
| | | also a receptor for |
| | | MADCAM1 and VCAM1. |
| | | It recognizes the sequence |
| | | L-D-T in MADCAM1. |
| | | Integrin alpha-E/beta-7 |
| | | (HML-1) is a receptor for |
| | | E-cadherin. |
| Integrin linked kinase | ↓ | Receptor-proximal protein |
| | | kinase regulating integrin- |
| | | mediated signal |
| | | transduction. May act as a |
| | | mediator of inside-out |
| | | integrin signaling. Focal |
| | | adhesion protein part of the |
| | | complex ILK-PINCH. This |
| | | complex is considered to be |
| | | one of the convergence |
| | | points of integrin- and |
| | | growth factor-signaling |
| | | pathway. Could be |
| | | implicated in mediating cell |
| | | architecture, adhesion to |
| | | integrin substrates and |
| | | anchorage-dependent |
| | | growth in epithelial cells. |
| | | Phosphorylates beta-1 and |
| | | beta-3 integrin subunit on |
| | | serine and threonine |
| | | residues, but also AKT1 |
| | | and GSK3B. |

Effect of Nutrition on Genes Involved with Muscle and Bone Regulation

Ten down regulated genes are identified as related to body composition through regulation of bone and muscle. The genes spare muscle and bone deterioration by reducing nitric oxide production and glucocorticoid degradation of muscle. Down regulation of these genes results in a decrease in nitric oxide production and glucocorticoid response. The compositions disclosed herein may be part of a therapeutic regimen to treat animals suffering from diseases or disorders associated with or relating to muscle or bone. These genes and their putative role in muscle and bone regulation are detailed in Tables 9 and 10 below.

TABLE 9
Genes involved in muscle and bone regulation

| | | | | | |
|---|---|---|---|---|---|
| | | | | % match of | |
| | | | | probe | |
| | | | Best current BLAST | sequence to | |
| Gene | Probe | P-value | annotation | BLAST hit | Probe Target Sequence |

| | | | | | |
|---|---|---|---|---|---|
| Capping | Cfa.1044.1. | 0.001 | PREDICTED: Canis | 44.87179 | AGGTCCCGTAACACCGGCAT |
| Protein | S1_at | | familiaris similar to F- | | CGCGACCGCACAGCGCCAT |
| | | | actin capping protein | | CTCCCCAGAATAAAGCCCAG |
| | | | beta subunit | | TAAACACCCCTGNNNNNNAN |
| | | | (LOC478209); mRNA | | NNNNANNNNNCACCACGTTT |
| | | | | | TGCTATCAGAACTCTCCTTGT |
| | | | | | TTCCAGAGCCCGTGTGCTTT |
| | | | | | TGTTTGCCCCAGCCCC |
| Calmodulin | Cfa.4168.1. | 0.01 | PREDICTED: Canis | 52.54237 | CCACCCATGGTGACGATGAC |
| | S1_at | | familiaris similar to | | ACACATCCTGGTGGCATGCG |
| | | | calmodulin 1; | | TGTGTTGGTTTAGCGTTGTCT |
| | | | transcript variant 3 | | GCGTTGTACTAGAGCGAAAA |
| | | | (LOC480416); mRNA | | TGGGTGTCAGGCTTGTCACC |
| | | | | | ATTCACACAGAAATTTAAAAA |
| | | | | | AAAAAAAAAAANNNNGANAA |
| | | | | | AAAACCTTTACCAAGGGAGC |
| | | | | | ATCTTTGGACTCTCTGTTTTT |
| | | | | | AAAACCTCCTGAACCATGAC |
| | | | | | TTGGAGCCAGCAGATTAGGC |
| | | | | | TGTGGCTGTGGACTTCAGCA |
| | | | | | CAACCATCAACATTGCTGATC |
| | | | | | AAGAAATTACAATATACGTCC |
| | | | | | ATTCCAAGTT |
| Dynein | Cfa.4942.1. | 0.001 | PREDICTED: Canis | 99.6016 | ATACCTCAGAGGTCTCGTAG |
| | A1_s_at | | familiaris similar to | | CTCGTGCCCTTGCCATCCAG |
| | | | dynein; cytoplasmic; | | AGCTGGGTGGNAGAGAGCT |
| | | | heavy polypeptide 2; | | GAGAAGCAGGCTCTTTTCTC |
| | | | transcript variant 2 | | TGATACACTCGACCTGTCAG |
| | | | (LOC479461); mRNA | | AACTCTTCCACCCAGACACA |
| | | | | | TTTCTCAATGCTCTTCGCCAG |
| | | | | | GAAACAGCAAGGGTGATGGG |
| | | | | | CTGCTCTGTGGATAGCCTTA |
| | | | | | AGTTTGTAGCTTCGTGGAAA |
| | | | | | GGTCGGCTGCAAGAAGCAAA |
| | | | | | GCTGCAGATCAAGATGGGCG |
| | | | | | GCTTGCTTCTGGAAGGCTGC |
| | | | | | AGTTTTGACGGGAGCCGGCT |
| | | | | | CTCTGAAAACCACCACGATT |
| | | | | | CTCCAAGTGTGTCACCAGTT |
| | | | | | CTCCCTTGCTGTGTTGGCTG |
| | | | | | GATTCCCCAGGGTGCATATG |
| | | | | | GTCCCTATTCTCCTGACGAG |
| | | | | | TGCATATCTCTGCCCGTGTA |
| | | | | | CACGAGCGCTGAGAGGGAT |
| | | | | | CGTGTGGTAGCCAACATCGA |
| | | | | | CGTCCCGTGTGGGGGCANC |
| | | | | | CAAGACCAGTGGATTCAGTG |
| | | | | | TGGAGCCGCTCTGTTTCTAA |
| | | | | | AAAA |
| Dynactin | Cfa.1807.1. | 0.01 | PREDICTED: Canis | 100 | AGGACGACAAGGCTCAGGAC |
| | S1_at | | familiaris similar to | | GCAAAGTGTGAAACTGCCTT |
| | | | dynactin 3 isoform 2; | | TGTAACAGGGCAGAAGCAGC |
| | | | transcript variant 1 | | TCTGTATTGGATTCACAACCT |
| | | | (LOC474750); mRNA | | ACCTATCTGCATTCAGGTGG |
| | | | | | GGCTCGGAGGTCAGAGGTCT |
| | | | | | GGCTACTTGAGGTTTGCTGT |
| | | | | | TTGCAC |
| Kinesin | Cfa.10496. | 0.01 | PREDICTED: Canis | 99.73046 | AGCCACAGCATTTCCTTTTAA |
| | 1.S1_s_at | | familiaris similar to | | CTTGGTTCAATTTTTGTAGCA |
| | | | Kinesin-like protein | | AGACTGAGCAGTTCTAAATC |
| | | | KIF2 (Kinesin-2) | | CTTTGCGTGCATGCATACCT |
| | | | (HK2); transcript | | CATCAGTGNACTGTACATAC |
| | | | variant 5 | | CTTGCCCTCTCCCAGAGACA |
| | | | (LOC478071); mRNA | | GCTGTGCTCACCTCTTCCTG |
| | | | | | CTTTGTGCCTTGACTAAGGC |
| | | | | | TTTTGACCCTAAATTTCTGAA |
| | | | | | GCACAGCCAAGATAAAGTAC |
| | | | | | ATTCCTTAATTGTCAGTGTAA |
| | | | | | ATTACCTTTATTGTGTGTACA |
| | | | | | TTTTTACTGTACTTGAGACAT |
| | | | | | TTTTTGTGTGTGACTAGTTAA |
| | | | | | TTTTGCAGGATGTGCCATATC |
| | | | | | ATTGAATGGAACTAAAGTCTG |
| | | | | | TGACAGTGGACATAGCTGCT |
| | | | | | GGACCATTCCATCTTACATGTA |
| Heat | CfaAffx.11022. | 0.01 | PREDICTED: Canis | 100 | GGTGCTACTGTTTGAAACAG |
| Shock | 1.S1_s_at | | familiaris similar to | | CTCTACTCTCCTCCGGCTTCT |
| Protein 1 | | | Heat shock protein | | CACTGGAGGATCCCCAGACT |
| (HSP90) | | | HSP 90-beta (HSP | | CACTCCAACCGCATTTACCG |
| | | | 84) (Tumor specific | | CATGATAAAGCTAGGCCTGG |
| | | | transplantation 84 kDa | | GCATCGATGAAGATGAAGTG |
| | | | antigen) (TSTA) | | GCAGCGGAGGAACCCAGTG |
| | | | (LOC611252); mRNA | | CTGCTGTTCCTGATGAGATC |
| | | | | | CCTCCACTTGAGGGTGATGA |
| | | | | | GGATGCCTCTCGCATGGAAG |
| | | | | | AAGTC |
| PPlase | CfaAffx.1740. | 0.01 | PREDICTED: Canis | 100 | GACATCACCAGTGGAGACGG |
| | 1.S1_at | | familiaris similar to | | CACCGGCGGTATAAGCATTT |
| | | | Peptidyl-prolyl cis- | | ATGGTGAGACGTTTCCAGAT |
| | | | trans isomerase C | | GAAAACTTCAAACTGAAGCAT |
| | | | (PPlase) (Rotamase) | | TATGGCATTGGTTGGGTCAG |
| | | | (Cyclophilin C) | | CATGGCCAACGCTGGGCCTG |
| | | | (LOC481480); mRNA | | ACACCAACGGCTCTCAGTTC |
| | | | | | TTTATCACCTTGACCAAGCCC |
| | | | | | ACTTGGTTGGATGGCAAACA |
| | | | | | TGTGGTATTTGGAAAAGTCCT |
| | | | | | TGATGGAATGACTGTGGTCC |
| | | | | | ACTCCATAGAACTTCAGGCA |
| | | | | | ACCGATGGGCACG |
| Calcinuerin | Cfa.19761. | 0.001 | PREDICTED: Canis | 98.83382 | GAATTAACAATCTGCTTGAGC |
| | 1.S1_at | | familiaris similar to | | CCCAAAACACTACTTATGCAC |
| | | | protein phosphatase | | TTCACTTGCCAAAAGATTTGN |
| | | | 3 (formerly 2B); | | GCAAGGTTTTGTACCCTGGT |
| | | | catalytic subunit; beta | | AAATGATGCCAAAGTTTGTTT |
| | | | isoform (calcineurin A | | TCTGTGGTGTTTGTCAAATGT |
| | | | beta); transcript | | TCTATGTATAATTGACTGTCT |
| | | | variant 5 | | GTAACATGCTGTTTNCTTCCT |
| | | | (LOC479248); mRNA | | CTGCAGATGTAGCTGCTTTC |
| | | | | | CTAAATCTGTCTGTCTTTCTT |
| | | | | | TAGGTTAGCTGTATGTCTGTA |
| | | | | | AAAGTATGTTAAATTAAATTA |
| | | | | | CTCTATCAGACGCTTGTCTGT |
| | | | | | CTTTTGATGTAGAAGCAACTT |
| | | | | | TGTAGCACCTTGTTTTGAGGT |
| | | | | | NNGCTGCATTTGTTGCTGTA |
| | | | | | CTTTGTGCAT |
| Protein | CfaAffx.408. | 0.01 | PREDICTED: Canis | 99.64664 | TTCAGTTCCTGTCTCATGGC |
| kinase C | 1.S1_s_at | | familiaris similar to | | CGCTCCCGGGACCATGCCAT |
| | | | myeloid-associated | | CGCCGCCACTGCCTTCTCCT |
| | | | differentiation marker | | GCATCGCTTGTGTGGCTTAT |
| | | | (LOC611521); mRNA | | GCCACCGAAGTGGCCTGGA |
| | | | | | CCCGGGCCCGTCCCGGAGA |
| | | | | | GATCACCGGCTACATGGCCA |
| | | | | | NTGTGCCGGGCCTGCTCAAG |
| | | | | | GTGCTGGAGACCTTTGTGGC |
| | | | | | CTGCATCATCTTCGCCTTCAT |
| | | | | | CAGCAACCCCTCCCTGTACC |
| | | | | | AGCACCAGCCGGCCCTGGA |
| | | | | | GTGGTGTGTGGCCGTCTACT |
| | | | | | CCATCTGTTTCATCCTGGCG |
| | | | | | GCTGTGGCCATCCTACTGAA |
| | | | | | CCTGGGGGACTGCACCAACA |
| | | | | | TGCTGCCCATCTCCTTCCCC |
| | | | | | AGTTTCCTGTCGGGCCTGGC |
| | | | | | CCTGCTCTCCGTCCTGCTGT |
| | | | | | ATGCCACGGCTCTGGNTCTC |
| | | | | | TGGCCGCTCTACCAGTTCAA |
| | | | | | CGAGAAGTATGGTGGCCAGC |
| | | | | | CCCGTCGGTCGAGGGATGTT |
| | | | | | AGCTGCGCCGACAGGCACA |
| | | | | | CCTACTACGTGTGTACCTGG |
| | | | | | GACCGCCGCCTGGCTGTGG |
| | | | | | CCATCCTGACAGCCATCAAC |
| | | | | | CTGCTGGCTTACGTGGCTGA |
| | | | | | CCTGGTGTAC |
| Protein | Cfa.15485. | 0.01 | PREDICTED: Canis | 100 | GGAGCAGTCAGAACTAAGAC |
| Kinase C | 1.A1_s_at | | familiaris similar to | | ATGGTCCGTTTTACTATATGA |
| Binding | | | protein kinase C | | AGCAGCCACTCACCACAGAC |
| Protein | | | binding protein 1 | | CCTGTTGATGTTGTACCGCA |
| | | | isoform b; transcript | | GGATGGACGGAA |
| | | | variant 11 | | |
| | | | (LOC477252); mRNA | | |

TABLE 10
Summary of genes affecting glucocorticoid receptors and nitric oxide
production

| | Gene | |
|---|---|---|
| | Expression | |
| | Compared | |
| Gene | to Control | Role |
| Kinesin | ↓ | Transport of organelles |
| | | from the (−) to (+) ends. |
| | | Binds microtubules. |
| | | ATPase activity |
| Capping Protein | ↓ | Part of dynactin-dynein |
| | | hetero-complex |
| Calmodulin | ↓ | Directly influences calcium |
| | | dependent dynein activity. |
| | | Binds to nitric oxide |
| | | synthase and up regulates |
| | | the production of nitric |
| | | oxide |
| Dynein | ↓ | Transport of organelles |
| | | from the (+) to (−) ends. |
| | | Binds microtubules. |
| | | ATPase activity and force |
| | | production |
| Dynactin | ↓ | Cytoplasmic dynein |
| | | activator. Binds |
| | | mirotubules and ↑average |
| | | length of dyein movements. |
| Heat Shock Protein 1 beta | ↓ | Necessary for |
| (HSP90) | | glucocorticoid receptor |
| | | binding and fast transport of |
| | | dynein complex to nucleus. |
| | | Calcinuerin activity. |
| | | Enhances the nitric oxide |
| | | production by binding to |
| | | nitric oxide synthase |
| PPIase | ↓ | Necessary for |
| | | dynein/glucocorticoid |
| | | interaction and movement |
| Calcinuerin | ↓ | Part of dynactin-dynein |
| | | hetero-complex. Catalyzes |
| | | the conversion of arginine |
| | | to citrulline and nitric oxide |
| Protein kinase C | ↓ | Calcium-activated, |
| | | phospholipid-dependent, |
| | | serine- and threonine- |
| | | specific enzyme. |
| Protein Kinase C Binding | ↓ | Associated with protein |
| Protein | | kinase C |

Effect of Nutrition on Genes Involved with DNA Damage/Protection and Neural Function

Eleven genes are identified that are related to DNA damage/protection and neural function. With regard to the latter, the genes identified are important for rebound potentiation; they are believed to have a potential role in motor learning. Interestingly, of these genes, all were down regulated except for of gamma-aminobutyric acid (GABA) A receptor, gamma 2 which was up regulated. The compositions disclosed herein may be part of a therapeutic regimen to treat animals suffering from diseases or disorders associated with or relating to DNA damage/protection and neural function. The identity of these genes and their putative role in DNA damage/protection and neural function are described in Tables 11 and 12 below.

TABLE 11
Genes involved in DNA damage/protection and neural function

| | | | | | |
|---|---|---|---|---|---|
| | | | | % match of | |
| | | | | probe | |
| | | | Best current | sequence to | Probe Target |
| Gene | Probe | P-value | BLAST annotation | BLAST hit | Sequence |

| | | | | | |
|---|---|---|---|---|---|
| Gamma- | CfaAffx.26362.1.S1_at | <0.01 | Homo sapiens | 100 | CCTCTTCTTCGGATGTTT |
| aminobutyric | | | gamma- | | TCCTTCAAGGCCCCTAC |
| acid (GABA) A | | | aminobutyric acid | | CATTGAT |
| receptor, | | | (GABA) A receptor; | | |
| gamma 2 | | | gamma 2 | | |
| | | | (GABRG2); | | |
| | | | transcript variant 1; | | |
| | | | mRNA | | |
| Calmodulin | Cfa.4168.1.S1_at | <0.01 | PREDICTED: | 52.54237 | CCACCCATGGTGACGAT |
| | | | Canis familiaris | | GACACACATCCTGGTGG |
| | | | similar to | | CATGCGTGTGTTGGTTT |
| | | | calmodulin 1; | | AGCGTTGTCTGCGTTGT |
| | | | transcript variant 3 | | ACTAGAGCGAAAATGGG |
| | | | (LOC480416); | | TGTCAGGCTTGTCACCA |
| | | | mRNA | | TTCACACAGAAATTTAAA |
| | | | | | AAAAAAAAAAAAANNNN |
| | | | | | GANAAAAAACCTTTACC |
| | | | | | AAGGGAGCATCTTTGGA |
| | | | | | CTCTCTGTTTTTAAAACC |
| | | | | | TCCTGAACCATGACTTG |
| | | | | | GAGCCAGCAGATTAGGC |
| | | | | | TGTGGCTGTGGACTTCA |
| | | | | | GCACAACCATCAACATT |
| | | | | | GCTGATCAAGAAATTAC |
| | | | | | AATATACGTCCATTCCAA |
| | | | | | GTT |
| Calcinuerin | Cfa.19761.1.S1_at | <0.001 | PREDICTED: | 98.83382 | GAATTAACAATCTGCTT |
| | | | Canis familiaris | | GAGCCCCAAAACACTAC |
| | | | similar to protein | | TTATGCACTTCACTTGC |
| | | | phosphatase 3 | | CAAAAGATTTGNGCAAG |
| | | | (formerly 2B); | | GTTTTGTACCCTGGTAA |
| | | | catalytic subunit; | | ATGATGCCAAAGTTTGT |
| | | | beta isoform | | TTTCTGTGGTGTTTGTCA |
| | | | (calcineurin A | | AATGTTCTATGTATAATT |
| | | | beta); transcript | | GACTGTCTGTAACATGC |
| | | | variant 5 | | TGTTTNCTTCCTCTGCA |
| | | | (LOC479248); | | GATGTAGCTGCTTTCCT |
| | | | mRNA | | AAATCTGTCTGTCTTTCT |
| | | | | | TTAGGTTAGCTGTATGT |
| | | | | | CTGTAAAAGTATGTTAAA |
| | | | | | TTAAATTACTCTATCAGA |
| | | | | | CGCTTGTCTGTCTTTTG |
| | | | | | ATGTAGAAGCAACTTTG |
| | | | | | TAGCACCTTGTTTTGAG |
| | | | | | GTNNGCTGCATTTGTTG |
| | | | | | CTGTACTTTGTGCAT |
| Calcium/calmodulin- | Cfa.3884.1.S1_at | <0.01 | Homo sapiens | 24.10714 | GGTGCTGTTCACCACAG |
| dependent | | | PTEN induced | | TAAGTGGCCTCTCAGTG |
| protein kinase | | | putative kinase 1 | | TTGCTGACCAAAGTGTG |
| II | | | (PINK1); mRNA | | AAATCCTAGAGCTTCAG |
| | | | | | GGGAGAGGACGTGGGG |
| | | | | | GAAATCCGGGGCTTGAC |
| | | | | | TTTATAATAGGATTATAG |
| | | | | | AGATGAAAAGTACACCT |
| | | | | | TGCTTTAGGCAACAGTT |
| | | | | | GGGATTCCTAAGACGCA |
| | | | | | TGTGTAAGAGCATATGT |
| | | | | | GAAATCCCTTCCCCATT |
| | | | | | GTTGATCTCTACTCACA |
| | | | | | GAATTTTGTCTTTATTAT |
| | | | | | GGTGTAAGAATCACTCT |
| | | | | | TAAAGCCACATATTCAAT |
| | | | | | TCAAAGCAAATACGTGT |
| | | | | | TCTGCAGTTGCAAATGT |
| | | | | | GTATTTAATTCTTCACAA |
| | | | | | TTCCTGTAAG |
| Adenylate | CfaAffx.5462.1.S1_s_at | <0.01 | PREDICTED: | 100 | GAAACTCGGTCTGGTGT |
| cyclase- | | | Canis familiaris | | TCGATGACGTCGTGGGC |
| associated | | | similar to Adenylyl | | ATTGTGGAGATAATCAA |
| protein 1 | | | cyclase-associated | | TAGTAGGGATGTCAAAG |
| | | | protein 1 (CAP 1); | | TTCAGGTAATGGGTAAA |
| | | | transcript variant 1 | | GTGCCAACCATTTCCAT |
| | | | (LOC475317); | | CAACAAAACAGATGGCT |
| | | | mRNA | | GCCATGTTTACCTGAGC |
| | | | | | AAGAATTCCCTGGATTG |
| | | | | | CGAAATAGTCAGTGCCA |
| | | | | | AATCTTCTGAGATGAAT |
| | | | | | GTCCTCATTCCTACTGA |
| | | | | | AGGCGGTGACTATAATG |
| | | | | | AATTCCCAGTCCCTGAG |
| | | | | | CAGTTCAAGACCCTATG |
| | | | | | GAATGGGCAGAAGTTGG |
| | | | | | TCACCACAGTGACAGAA |
| | | | | | ATTGCTGGATAAGCGAA |
| | | | | | GTGCCACTGGGTTCTTT |
| | | | | | GCCCTCCCCCTCACACC |
| | | | | | ATGGGATAAATCTATCA |
| | | | | | GGACGGTTCTTTTCTAG |
| | | | | | ATTTCCTTTACCTTTCTG |
| | | | | | CTCTTAAACTGCTT |
| Protein | Cfa.6174.1.A1_at | <0.01 | PREDICTED: | 100 | AAATCTTACGAAGCCCA |
| Phosphatase I | | | Canis familiaris | | ATATGCAGGGAGTTAAC |
| | | | similar to protein | | TGAAAACTATCTTGGCA |
| | | | phosphatase 1A | | GTGAGGTTGGCACTGTT |
| | | | isoform 1; | | GATAAAGCTGGTCCCTT |
| | | | transcript variant 2 | | CCTTTAACTGTCTTTTAG |
| | | | (LOC480344); | | GTTGTTCTTGCCTTGTT |
| | | | mRNA | | GCCAGGAGTATTGCAGG |
| | | | | | TAATACAGTATATTCATA |
| | | | | | AGAATATCAATCTTGGG |
| | | | | | GCTAAAATGCCTTGATT |
| | | | | | CTTTGCACCTCTTTTACA |
| | | | | | AGTCCTTACGTTGAATTA |
| | | | | | CTAATTGATAAGCAGCA |
| | | | | | GCTTCCTACATATAGTA |
| | | | | | GGAGACTGCCACGTTTT |
| | | | | | TGCTATCATGATTGGCT |
| | | | | | GGGCCTGCTGCTGTTCC |
| | | | | | TAGTAAGGTAT |
| Diazepam | CfaAffx.14836.1.S1_s_at | <0.01 | PREDICTED: | 100 | AATGGTGCCATCTTACT |
| binding | | | Canis familiaris | | GAGGGATTTTGTAGGCT |
| inhibitor | | | similar to | | GTTTTATAGATTTTCCTA |
| | | | peroxisomal | | AGCCTCTGGTTGCAGTG |
| | | | D3; D2-enoyl-CoA | | ATAAATGGTCCAGCCAT |
| | | | isomerase isoform | | AGGAATCTCCGTCACCA |
| | | | 1 (LOC478706); | | TTCTCGGGCTATTCGAT |
| | | | mRNA | | CTTGTGTATGCTTCCGA |
| | | | | | CAGGGCAACATTTCACA |
| | | | | | CTCCTTTTACTCACCTG |
| | | | | | GGCCAAAGTCCAGAAG |
| | | | | | GATGTTCCTCCTATACTT |
| | | | | | TTCCCAAGATAATGGGC |
| | | | | | CAAGCCAAGGCAGCAG |
| | | | | | AGATGCTCATGTTTGGA |
| | | | | | AAGAAGTTAACAGCTAG |
| | | | | | AGAAGCCTGTGCTCAAG |
| | | | | | GACTTGTTACTGAAGTTT |
| | | | | | TTCCCGATAGCACTTTT |
| | | | | | CAGAAAGAAGTTTGGAC |
| | | | | | CAGGCTGAAAGCATATT |
| | | | | | CAAAACTCCCCCGAAAT |
| | | | | | ACCTTGCATATTTCCAAA |
| | | | | | CAGAGCATCAGAAATCT |
| | | | | | TGAGAAAGAAAAGCTAC |
| | | | | | ATGCTGTTAACGCAGAA |
| | | | | | GAAAACAGCGTCCTCCA |
| | | | | | GGAAAGGTGGCTGTCA |
| | | | | | GACGAATGCATAAATGC |
| | | | | | AGTCATGAGCTTCTTAT |
| | | | | | CCCGGAAGGCCAA |
| Tumor protein | Cfa.1611.1.A1_s_at | <0.01 | PREDICTED: | 97.90874 | ATGATAGTTGCCATGCC |
| p53 binding | | | Canis familiaris | | AACCAGCTCCAGAATTA |
| protein | | | similar to tumor | | CCGCAATTATTTGTTGC |
| | | | protein p53 binding | | CTGCAGGGTACAGCCTT |
| | | | protein; 1; | | GAGGAGCAAAGAATTCT |
| | | | transcript variant 4 | | GGATTGGCAACCCCGTG |
| | | | (LOC478274); | | AAAACCCTTTCCACAAT |
| | | | mRNA | | CTGAAGGTACTCTTGGT |
| | | | | | GTCAGACCAACAGCAGA |
| | | | | | ACTTCCTGGAGCTCTGG |
| | | | | | TCTGAGATCCTCATGAC |
| | | | | | CGGGGGGGCAGCCTCT |
| | | | | | GTGAAGCAGCACCATTC |
| | | | | | AAGTGCCCATAACAAAG |
| | | | | | ATATTGCTTTAGGGGTA |
| | | | | | TTTGACGTGGTGGTGAC |
| | | | | | GGATCCCTCATGCCCAG |
| | | | | | CCTCGGTGCTGAAGTGT |
| | | | | | GCTGAAGCATTGCAGCT |
| | | | | | GCCTGTGGTGTCACAAG |
| | | | | | AGTGGGTGATCCAGTGC |
| | | | | | CTCATTGTTGGGGAGAG |
| | | | | | AATTGGATTCAAGCAGC |
| | | | | | ATCCAAAATACAAACAT |
| | | | | | GATTATGTTTCTCACTAA |
| | | | | | TACTTGGTCTTAACTGAT |
| | | | | | TTTATTCCCTGCTGTTGT |
| | | | | | GGAGATTGTGNTTNNNC |
| | | | | | CAGGTTTTAAATGTGTCT |
| | | | | | TGTGTGTAACTGGATTC |
| | | | | | CTTGCATGGATCT |
| Ubiquitin | CfaAffx.275.1.S1_s_at | <0.001 | PREDICTED: Pan | 97.19626 | GATTTGGCCCGTGACCC |
| conjugating | | | troglodytes | | TCCAGCACAATGTTCTG |
| enzyme E2D 3 | | | LOC461941 | | CAGGTCCTGTTTGGGAT |
| | | | (LOC461941); | | GATATGTTTCATTGGCA |
| | | | mRNA | | AGCCACAATTATAGGAC |
| | | | | | CTAATGACAGCCCATAT |
| | | | | | CAAGG |
| NEDD8 | Cfa.12556.1.A1_s_at | <0.001 | PREDICTED: | 99.12473 | GGAATGGGCTACTCTAC |
| ultimate | | | Canis familiaris | | TCATGCAGNCAAGCAGG |
| buster-1 | | | similar to NEDD8 | | NCCTGCATCAGGCCAGT |
| | | | ultimate buster-1 | | GGGAACCTGGACGAAG |
| | | | (NY-REN-18 | | CCCTGAAGATTCTTCTC |
| | | | antigen) | | AGCAATCCTCAGATGTG |
| | | | (LOC475542); | | GTGGTTAAATGATTCAG |
| | | | mRNA | | ATCCTGAAACGANCAAC |
| | | | | | CAGCAAGAAAGTCCTTC |
| | | | | | CCAGGAAAACATTGACC |
| | | | | | AACTGGTGTACATGGGC |
| | | | | | TTCGACGCTGTGGTGGC |
| | | | | | TGATGCTGCCTTGAGAG |
| | | | | | TGTTCAGGGGAAACGTG |
| | | | | | CAGCTGGCAGCTCAGN |
| | | | | | CCCTCGCCCACAACGGA |
| | | | | | GGAACTCTTCCTCCTGA |
| | | | | | CCTGCAGCTCTTGGTGG |
| | | | | | AAGACTCTTCATCAACG |
| | | | | | CCATCCACGTCCCCTTC |
| | | | | | CGACTCCGCAGGTACCT |
| | | | | | CTAGTGCCTCAACAGAT |
| | | | | | GAAGATATGGAAACCGA |
| | | | | | AGCTGTCAATGAAATAC |
| | | | | | TGGAAGATATTCCAGAA |
| | | | | | CATGAAGAAGATTATCTT |
| | | | | | GACTCAACACTGGAAG |
| BCL2- | CfaAffx.6742.1.S1_s_at | <0.01 | Canis familiaris | 100 | GGCCCACCAGCTCTGA |
| associated X | | | BCL2-associated X | | GCAGATCATGAAGACAG |
| protein (BAX) | | | protein (BAX); | | GGGCCCTTTTGCTTCAG |
| | | | mRNA | | GGTTTCATCCAAGATCG |
| | | | | | AGCAGGGCGAATGGGG |
| | | | | | GGAGAGACACCTGAGCT |
| | | | | | GCCCTTGGAGCAGGTG |
| | | | | | CCCCAGGATGCATCCAC |
| | | | | | CAAGAAGCTGAGCGAAT |
| | | | | | GTCTCAAGCGCATCGGA |
| | | | | | GATGAACTGGACAGTAA |
| | | | | | CATGGAGTTGCAGAGGA |
| | | | | | TGATCGCAGCTGTGGAC |
| | | | | | ACAGACTCTCCCCGTGA |
| | | | | | GGTCTTCTTCCGAGTGG |
| | | | | | CAGCTGAGATGTTTTCT |
| | | | | | GATGGCAACTTCAACTG |
| | | | | | GGGCCGGGTTGTTGCC |
| | | | | | CTCTTCTACTTTGCCAG |
| | | | | | CAAACTGGTGCTCA |

TABLE 12
Summary of genes important for rebound potentiation
and DNA integrity

| | Gene | |
|---|---|---|
| | Expression | |
| | Compared | |
| Gene | to Control | Role |
| Gamma-aminobutyric acid | ↑ | Involved in single channel |
| (GABA) A receptor, | | conductance (Cl-channel) |
| gamma 2 | | |
| Calmodulin | ↓ | Influx of calcium results in |
| | | calcium/calmodulin |
| | | complex which activates |
| | | CaMKII and calcineurin |
| Calcinuerin | ↓ | Involved in the pathway for |
| | | RP suppression |
| Calcium/calmodulin- | ↓ | Involved in induction and |
| dependent protein kinase II | | suppression of RP |
| Adenylate cyclase- | ↓ | Adenlyl cyclase is involved |
| associated protein 1 | | in suppression of RP |
| Protein Phosphatase I | ↓ | Dephosphorylates |
| | | components in stress- |
| | | activated pathways. Active |
| | | PP-1 results in CaMKII |
| | | inhibition and RP |
| | | suppression |
| Diazepam binding inhibitor | ↓ | Displaces benzodiazepine |
| | | Down regulates the effects |
| | | of GABA |
| Tumor protein p53 binding | ↓ | Keep the cell from |
| protein | | progressing through the cell |
| | | cycle if there is damage to |
| | | DNA present. |
| Ubiquitin conjugating | ↓ | The regulated proteolysis of |
| enzyme E2D 3 | | proteins by proteasomes |
| (and NEDD8 ultimate | | removes denatured, |
| buster-1) | | damaged or improperly |
| | | translated proteins from |
| | | cells and regulates the level |
| | | of proteins like cyclins or |
| | | some transcription factors |
| BCL2-associated X protein | ↓ | Accelerates programmed |
| | | cell death by binding to, and |
| | | antagonizing the apoptosis |
| | | repressor |
| | | BCL2 |

Effect of Nutrition on Genes Involved with Glucose Metabolism

Twenty four genes associated with glucose metabolism are down regulated in animals fed the super senior diet which would suggest that these animals are utilizing fat (fat oxidation) instead of glucose as a fuel source. The compositions disclosed herein may be part of a therapeutic regime in diabetic animals and/or for obesity prevention or treatment in an animal. These down regulated genes are identified and their putative role in glucose metabolism described in detail below in Tables 13 and 14.

TABLE 13
Genes involved in Glucose Metabolism

| | | | | | |
|---|---|---|---|---|---|
| | | | | % match of | |
| | | | | probe | |
| | | | Best current | sequence to | Probe Target |
| Gene | Probe | P-value | BLAST annotation | BLAST hit | Seq. |

| | | | | | |
|---|---|---|---|---|---|
| Phosphorylase | Cfa.10856.1.S1_at | <0.01 | PREDICTED: Canis | 99.3392 | GAAAGTTCACCA |
| kinase | | | familiaris similar to | | CTGCATGTTTTAT |
| | | | phosphorylase kinase | | GATCAGATAACT |
| | | | beta; transcript variant | | CATTGAAATGAG |
| | | | 2 (LOC478139); | | TCTTTGCTCTTTA |
| | | | mRNA | | GACTAAATTCCC |
| | | | | | ACCTAGTACTGC |
| | | | | | CATTAAAATGAAT |
| | | | | | TTGCCAGCTGGT |
| | | | | | GTGCATACTGGA |
| | | | | | AATGAAAAGATA |
| | | | | | CTGAAAGAATGG |
| | | | | | AACGAATGGTGA |
| | | | | | GCTTAACTCAGT |
| | | | | | GGCACTGTCATA |
| | | | | | CTGGAAAAATAC |
| | | | | | AGTAAAATCATAA |
| | | | | | AAACAGATCTGC |
| | | | | | CAGCTGATGTTT |
| | | | | | TTATTCTCAGAAA |
| | | | | | CAGCATTGTTGA |
| | | | | | TAATATTTTAGTA |
| | | | | | TACAGAGCTACT |
| | | | | | GTACAATTTTTAC |
| | | | | | CTTGNAAACATG |
| | | | | | ACTGTGGTTTTG |
| | | | | | TATTTGTGTTGAC |
| | | | | | TTTAGGGGTTGG |
| | | | | | GATAAAATNCAG |
| | | | | | TATAATATATACC |
| | | | | | TTATCAAACNTTT |
| | | | | | TCTTTGAGCTCTT |
| | | | | | ACTAAAAATATG |
| | | | | | GCATGCATAAGA |
| | | | | | TTGTTCAGAAGA |
| | | | | | GTAGACTGTTAA |
| | | | | | CCTAGTTTGTA |
| Phosphorylase | Cfa.10412.1.A1_s_at | <0.01 | PREDICTED: Canis | 99.36306 | CTTCCAGAGCTG |
| | | | familiaris | | AAGCTGGCCATT |
| | | | phosphorylase; | | GATCNAAATTGA |
| | | | glycogen; liver; | | CAATGGCTTCTT |
| | | | transcript variant 1 | | CTCTCCCAAGCA |
| | | | (PYGL); mRNA | | GCCTGNCCTCTT |
| | | | | | CAAAGATTTAATC |
| | | | | | AATATGCTATTTT |
| | | | | | ATCATGACAGGT |
| | | | | | TTAAAGTCTTCG |
| | | | | | CAGACTATGAAG |
| | | | | | CCTATGTCAAGT |
| | | | | | GTCAAGAAAAAG |
| | | | | | TCAGCCAGCTGT |
| | | | | | ACATGAATCCAA |
| | | | | | AGGCCTGGAACA |
| | | | | | CAATGGTACTCA |
| | | | | | AAAACATAGCTG |
| | | | | | CCGCAGGGAAGT |
| | | | | | TCTCTAGTGACC |
| | | | | | GAACAATTAAGG |
| | | | | | AATATGCCAGGG |
| | | | | | ACATCTGGAACA |
| | | | | | TGGAACCTTCAG |
| | | | | | ATCTCAAGATTTC |
| | | | | | CCTATCCAATG |
| Glycogen | Cfa.913.1.A1_s_at | <0.01 | PREDICTED: Canis | 99.49622 | GACTCCACCGGA |
| synthase kinase 3 | | | familiaris similar to | | GGCAATTGCACT |
| | | | Glycogen synthase | | GTGTAGCCGTCT |
| | | | kinase-3 beta (GSK-3 | | GCTGGAGTATAC |
| | | | beta); transcript | | ACCAACTGCCCG |
| | | | variant 1 | | ATTGACACCACT |
| | | | (LOC478575); mRNA | | GGAAGCTTGTGC |
| | | | | | ACATTCATTTTTT |
| | | | | | GATGAATTAAGG |
| | | | | | GACCCAAATGTC |
| | | | | | AAACTACCAAAT |
| | | | | | GGGCGAGACACA |
| | | | | | CCTGCACTCTTC |
| | | | | | AACTTCACCACT |
| | | | | | CAAGAACTGTCA |
| | | | | | AGTAATCCACCT |
| | | | | | CTAGCTACCATC |
| | | | | | CTTATTCCTCCTC |
| | | | | | ATGCTCGGATTC |
| | | | | | AAGCAGCTGCTT |
| | | | | | CAACCCCTACAA |
| | | | | | ATGCCACAGCAG |
| | | | | | CCTCAGATGCTA |
| | | | | | ATGCCGGAGACC |
| | | | | | GTGGACAGACGA |
| | | | | | ACAATGCCNCTT |
| | | | | | CTGCATCAGCTT |
| | | | | | CTAACTCCACCT |
| | | | | | GAACAGTCCCGA |
| | | | | | GCAGCCAGCTGC |
| | | | | | ACAGGAAGAACC |
| | | | | | ACCAGTTACTTG |
| | | | | | AGTGTCACTCA |
| Calmodulin | Cfa.4168.1.S1_at | <0.01 | PREDICTED: Canis | 52.54237 | CCACCCATGGTG |
| | | | familiaris similar to | | ACGATGACACAC |
| | | | calmodulin 1; | | ATCCTGGTGGCA |
| | | | transcript variant 3 | | TGCGTGTGTTGG |
| | | | (LOC480416); mRNA | | TTTAGCGTTGTCT |
| | | | | | GCGTTGTACTAG |
| | | | | | AGCGAAAATGGG |
| | | | | | TGTCAGGCTTGT |
| | | | | | CACCATTCACAC |
| | | | | | AGAAATTTAAAAA |
| | | | | | AAAAAAAAAAAN |
| | | | | | NNNGANAAAAAA |
| | | | | | CCTTTACCAAGG |
| | | | | | GAGCATCTTTGG |
| | | | | | ACTCTCTGTTTTT |
| | | | | | AAAACCTCCTGA |
| | | | | | ACCATGACTTGG |
| | | | | | AGCCAGCAGATT |
| | | | | | AGGCTGTGGCTG |
| | | | | | TGGACTTCAGCA |
| | | | | | CAACCATCAACA |
| | | | | | TTGCTGATCAAG |
| | | | | | AAATTACAATATA |
| | | | | | CGTCCATTCCAA |
| | | | | | GTT |
| Protein Kinase C | CfaAffx.408.1.S1_s_at | <0.01 | PREDICTED: Canis | 99.64664 | TTCAGTTCCTGT |
| | | | familiaris similar to | | CTCATGGCCGCT |
| | | | myeloid-associated | | CCCGGGACCATG |
| | | | differentiation marker | | CCATCGCCGCCA |
| | | | (LOC611521); mRNA | | CTGCCTTCTCCT |
| | | | | | GCATCGCTTGTG |
| | | | | | TGGCTTATGCCA |
| | | | | | CCGAAGTGGCCT |
| | | | | | GGACCCGGGCC |
| | | | | | CGTCCCGGAGAG |
| | | | | | ATCACCGGCTAC |
| | | | | | ATGGCCANTGTG |
| | | | | | CCGGGCCTGCTC |
| | | | | | AAGGTGCTGGAG |
| | | | | | ACCTTTGTGGCC |
| | | | | | TGCATCATCTTC |
| | | | | | GCCTTCATCAGC |
| | | | | | AACCCCTCCCTG |
| | | | | | TACCAGCACCAG |
| | | | | | CCGGCCCTGGA |
| | | | | | GTGGTGTGTGGC |
| | | | | | CGTCTACTCCAT |
| | | | | | CTGTTTCATCCT |
| | | | | | GGCGGCTGTGG |
| | | | | | CCATCCTACTGA |
| | | | | | ACCTGGGGGACT |
| | | | | | GCACCAACATGC |
| | | | | | TGCCCATCTCCT |
| | | | | | TCCCCAGTTTCC |
| | | | | | TGTCGGGCCTGG |
| | | | | | CCCTGCTCTCCG |
| | | | | | TCCTGCTGTATG |
| | | | | | CCACGGCTCTGG |
| | | | | | NTCTCTGGCCGC |
| | | | | | TCTACCAGTTCA |
| | | | | | ACGAGAAGTATG |
| | | | | | GTGGCCAGCCCC |
| | | | | | GTCGGTCGAGG |
| | | | | | GATGTTAGCTGC |
| | | | | | GCCGACAGGCAC |
| | | | | | ACCTACTACGTG |
| | | | | | TGTACCTGGGAC |
| | | | | | CGCCGCCTGGCT |
| | | | | | GTGGCCATCCTG |
| | | | | | ACAGCCATCAAC |
| | | | | | CTGCTGGCTTAC |
| | | | | | GTGGCTGACCTG |
| | | | | | GTGTAC |
| Protein Kinase C | Cfa.15485.1.A1_s_at | <0.01 | PREDICTED: Canis | 100 | GGAGCAGTCAGA |
| Binding Protein | | | familiaris similar to | | ACTAAGACATGG |
| | | | protein kinase C | | TCCGTTTTACTAT |
| | | | binding protein 1 | | ATGAAGCAGCCA |
| | | | isoform b; transcript | | CTCACCACAGAC |
| | | | variant 11 | | CCTGTTGATGTT |
| | | | (LOC477252); mRNA | | GTACCGCAGGAT |
| | | | | | GGACGGAA |
| Hexokinase 3 | Cfa.19125.2.S1_at | <0.01 | Macaca fascicularis | 76.70683 | TAATGACTGCCA |
| | | | testis cDNA; clone: | | ACTCACTGTTTGT |
| | | | QtsA-14856; similar to | | TGGAGTTATATG |
| | | | human receptor | | CAGAAATAAAGN |
| | | | associated protein 80 | | CCAAGTCTTCAG |
| | | | (RAP80); mRNA; | | AAACAGGCTTCA |
| | | | RefSeq: | | GGATGCCCTCAC |
| | | | NM_016290.3 | | CAGGGATGGAAG |
| | | | | | AGGCAGGCTGCA |
| | | | | | GCAAAGAGATGC |
| | | | | | AGAGTTCCCTTG |
| | | | | | CACATCTCGACT |
| | | | | | TAAATGAGTCTC |
| | | | | | CCATCAAGTCTTT |
| | | | | | TGTTTCCATTTCA |
| | | | | | GAAGCCACAGAT |
| | | | | | TGCTTAGTGGAC |
| | | | | | TTTAAAAAGCAAC |
| | | | | | TTAACGTTCGGC |
| | | | | | AAGGTAGTCGGA |
| | | | | | CACGGACCAAAG |
| | | | | | CAGGCAGAGGAA |
| | | | | | GAAGGAGAAAAC |
| | | | | | CCTGAATTTCTA |
| | | | | | GGGTCCAGACAC |
| | | | | | CCGACAAAACCA |
| | | | | | TTAGCAATAGGG |
| | | | | | GTGGGCCGTGTC |
| | | | | | ATTAAGTCTTAGT |
| | | | | | GGCTTCTGTTTC |
| | | | | | ATTGTTGAACAA |
| | | | | | GTTTTTTGGCCC |
| | | | | | NGCAGTTTTCAC |
| | | | | | CACCAGCACCAA |
| | | | | | CTCAGCATTCTT |
| | | | | | GTTTTGATGTTTT |
| | | | | | CTATAAGCTATAC |
| | | | | | AGACAATTGTGT |
| | | | | | ATAGTATTCTGTT |
| | | | | | TTATAACAGTCTG |
| | | | | | GATTCACTT |
| Fructose 1,6 | CfaAffx.26135.1. | <0.01 | PREDICTED: Canis | 100 | AGTGGCGCTGTG |
| bisphosphatase | S1_s_at | | familiaris aldolase A; | | TGCTGAAAATTG |
| | | | transcript variant 1 | | GGGAACACACTC |
| | | | (LOC479787); mRNA | | CCTCAGCCCTTG |
| | | | | | CGATCATGGAAA |
| | | | | | ATGCCAACGTTC |
| | | | | | TGGCCCGTTAT |
| Glyceraldehyde | AFFX- | <0.01 | Canis familiaris | 100 | AGCTCACTGGCA |
| 3-phosphate | Cf_Gapdh_3_at | | glyceraldehyde-3- | | TGGCCTTCCGTG |
| dehydrogenase | | | phosphate | | TCCCCACCCCCA |
| | | | dehydrogenase | | ATGTATCAGTTGT |
| | | | (GAPDH); mRNA | | GGATCTGACCTG |
| | | | | | CCGCCTGGAGAA |
| | | | | | AGCTGCCAAATA |
| | | | | | TGACGACATCAA |
| | | | | | GAAGGTAGTGAA |
| | | | | | GCAGGCATCGGA |
| | | | | | GGGACCCCTCAA |
| | | | | | AGGCATCCTGGG |
| | | | | | CTACACTGAGGA |
| | | | | | CCAGGTGGTCTC |
| | | | | | CTGTGACTTCAA |
| | | | | | CAGTGACACCCA |
| | | | | | CTCTTCCACCTT |
| | | | | | CGACGCCGGGG |
| | | | | | CTGGCATTGCCC |
| | | | | | TCAATGACCACT |
| | | | | | TTGTCAAGCTCA |
| | | | | | TTTCCTGGTATG |
| | | | | | ACAATGAATTTG |
| | | | | | GCTACAGCAACC |
| | | | | | GGGTGGTGGAC |
| | | | | | CTCATGGTCTAC |
| | | | | | ATGG |
| Glucose 6- | Cfa.19351.1.S1_at | <0.01 | Homo sapiens cDNA | 15.11194 | GAATGTGTTGGC |
| phosphate | | | FLJ30869 fis; clone | | AGACTGAGGCCC |
| dehydrogenase | | | FEBRA2004224 | | CCCATGTTTTTAA |
| | | | | | TGCGCACTGGGG |
| | | | | | ACAACCATCTAA |
| | | | | | GGTCTAGAAACT |
| | | | | | TTTGGACCATAG |
| | | | | | GAAAGATAGGTT |
| | | | | | TATGGTCCTCTT |
| | | | | | CCAGATGCAGCC |
| | | | | | CTAGGAGAGCAT |
| | | | | | TCCCATGGGGTC |
| | | | | | TCTGGATCCCTT |
| | | | | | TCNTTGCTCTGT |
| | | | | | GAGGCTCTGTGA |
| | | | | | CCACCTTTTGNN |
| | | | | | NTGNNGGGGGC |
| | | | | | AGGGGGNCTTCC |
| | | | | | TCAGCTCCGCCT |
| | | | | | CCAGTGCCCCCA |
| | | | | | GGTCCCCCACGG |
| | | | | | CTCACAGTCCNT |
| | | | | | GAAAATTCAGAG |
| | | | | | CTGCCCTGTAAG |
| | | | | | GATTTTGTCCACT |
| | | | | | GGGCAATTCAGA |
| | | | | | TATACTTCGATAT |
| | | | | | CCCTGAGAAAGA |
| | | | | | AGAGGCAGCAGC |
| | | | | | AAACACTCCCNA |
| | | | | | GGGCATCTGTCT |
| | | | | | CAGNANTCTCTC |
| | | | | | NTTGNATGAGAC |
| | | | | | AGAAGCCTACTT |
| | | | | | TTCAGAAANCTTA |
| | | | | | TCANGGNTACTT |
| | | | | | TATAAGAAACTTT |
| | | | | | TTTTTTTTTNCTA |
| | | | | | AAATCAGACAAA |
| | | | | | AGGTGGCTTNTG |
| | | | | | CATATTCTTNATT |
| | | | | | AATAACTGTGTCT |
| | | | | | TTGTCTCCTCTG |
| | | | | | CTTAACTTTAGGA |
| Enolase | CfaAffx.30133.1. | <0.01 | PREDICTED: Canis | 97.72257 | GGTACATCACGC |
| | S1_s_at | | familiaris similar to | | CTGATCAGCTGG |
| | | | T21B10.2b; transcript | | CTGACCTCTACA |
| | | | variant 1 | | AGTCCTTCATCA |
| | | | (LOC479597); mRNA | | GGGACTACCCAG |
| | | | | | TGGTGTCTATCG |
| | | | | | AAGACCCCTTCG |
| | | | | | ACCAGGATGACT |
| | | | | | GGGAAGCTTGGC |
| | | | | | AGAAATTCACTG |
| | | | | | CCAGCGCTGGAA |
| | | | | | TCCAGGTGGNGG |
| | | | | | GGGANGATCTCA |
| | | | | | CCGTGACCAACC |
| | | | | | CAAAGCGGATTT |
| | | | | | CCAAGGCTGTGG |
| | | | | | GCGAGAAATNGT |
| | | | | | GCAACTGCCTCC |
| | | | | | TGCTTAAAGTGA |
| | | | | | ACCAGATTGGCT |
| | | | | | CTGTGACCGAGT |
| | | | | | CTCTTCAGGCGT |
| | | | | | GCAAGCTGGCCC |
| | | | | | AGTCCAATGGGT |
| | | | | | GGGGCGTCATG |
| | | | | | GTGTCGCATCGC |
| | | | | | TCCGGGGAGACC |
| | | | | | GAAGATACCTTC |
| | | | | | ATCGCTGACCTG |
| | | | | | GTGGTGGGANTC |
| | | | | | TGCACTGGGCAG |
| | | | | | ATCAAGACGGGT |
| | | | | | GCACCATGCAGA |
| | | | | | TCTGAGCGCTTG |
| | | | | | GCCAAGTACAAC |
| | | | | | CAGATCCTCAGA |
| | | | | | ATTGAAGAGGAA |
| | | | | | CTGGGTAGCAAG |
| | | | | | GCCAAGTTCGCC |
| | | | | | GGCAGAAGCTTC |
| | | | | | AGAA |
| Lactate | Cfa.300.1.S1_at | <0.01 | PREDICTED: Canis | 97.99427 | ATCTGACCTGTT |
| dehydrogenase | | | familiaris similar to L- | | ACTCAAGTCGTA |
| | | | lactate | | ATATTAAAATGGC |
| | | | dehydrogenase A | | CTAAGAAAAAAA |
| | | | chain (LDH-A) (LDH | | CATCAGTTTCCTA |
| | | | muscle subunit) | | AAGTTACACATA |
| | | | (LDH-M) | | GGAATGGTTCAC |
| | | | (Proliferation-inducing | | AAAACCCTGCAG |
| | | | gene 19 protein); | | CTATGTCCTGAT |
| | | | transcript variant 1 | | GCTGGATGAGAC |
| | | | (LOC476882); mRNA | | CTGTCTTGTGTA |
| | | | | | GTCCTAAATTGG |
| | | | | | TTAACGTAATATC |
| | | | | | GGAGGCACCACT |
| | | | | | GCCAATGTCATA |
| | | | | | TATGCTGCAGCT |
| | | | | | ACTCCTTAAACC |
| | | | | | AGATGTGTATTTA |
| | | | | | CTGTGTTTTGTAA |
| | | | | | CTTCTGATTCCTT |
| | | | | | CATCCCAACATC |
| | | | | | CAACATGCCTAG |
| | | | | | GCCATCTTTTCTT |
| | | | | | CTTCAGTCACAT |
| | | | | | CCTGGGATCCAA |
| | | | | | TGTATAAATTCAA |
| | | | | | TATTGCATGTATT |
| | | | | | GTGCATAACTCT |
| | | | | | TCTA |
| Citrate lyase | Cfa.10361.2.S1_at | <0.01 | PREDICTED: Canis | 98.49624 | AGTATGCCAGAT |
| | | | familiaris similar to | | CGGAACCTTTTT |
| | | | citrate lyase beta like | | CCCATTTACAGTT |
| | | | (LOC476974); mRNA | | CATGTTAATCCAA |
| | | | | | TTTTTTTTATTAT |
| | | | | | CTCACTGGCCAG |
| | | | | | TTATTCCTTTAAA |
| | | | | | AATGAACTTCCTT |
| | | | | | CTTTTTGATTCCA |
| | | | | | AGCTTATGATTTT |
| | | | | | ACTGCTCATTAAT |
| | | | | | GTGTTACAAATAT |
| | | | | | GCACTTAATGATT |
| | | | | | TCACAGGGAGAT |
| | | | | | AAAATAGTGAAG |
| | | | | | AGAGATGGGCTG |
| | | | | | AGGGGCTGTTAG |
| | | | | | GACTTTAATGAAA |
| | | | | | CAGATCTTTCCC |
| | | | | | GAATATTTCTCCC |
| | | | | | TTCACATTTCTCA |
| | | | | | CATTAGATGTTTC |
| | | | | | CCACATTGTTCTA |
| | | | | | CTCCACACTATA |
| | | | | | AATAATTTTAAGG |
| | | | | | CCAATCTTAAAAA |
| | | | | | ATGGTAGTTAAG |
| | | | | | TGAAGGGGTTGT |
| | | | | | GTTTATTTCACTA |
| | | | | | GAAATCTGATAA |
| | | | | | AACGAGAGATGA |
| | | | | | CATAGAAAAAGT |
| | | | | | TATCATTTTTGTT |
| | | | | | CATACAGATGGC |
| | | | | | TTCTAAAAATAAA |
| | | | | | TCTTCAAAACTGA |
| | | | | | TTACTTTTAACCT |
| | | | | | CCACCTCCCAAA |
| | | | | | ATGAAACATCCC |
| | | | | | TACATTTGAACTG |
| | | | | | CTAGGTGAGAAC |
| | | | | | TCTGAAAGCCCT |
| | | | | | CATCC |
| Glycerol kinase | CfaAffx.21204.1. | <0.01 | PREDICTED: Canis | 100 | GGGTACATCCTA |
| | S1_s_at | | familiaris similar to | | TGGCTGCTATTT |
| | | | glycerol kinase | | CGTCCCCGCGTT |
| | | | isoform 2; transcript | | TTCAGGGTTATAT |
| | | | variant 8 | | GCACCTTACTGG |
| | | | (LOC480872); mRNA | | GAGCCCAGTGCA |
| | | | | | AGAGGGATCATC |
| | | | | | TGTGGGCTCACT |
| | | | | | CAATTCACCAATA |
| | | | | | AATGCCATATTG |
| | | | | | CTTTTGCTGCATT |
| | | | | | AGAAGCTGTTTG |
| | | | | | TTTCCAAACCCG |
| | | | | | GGAGATTTTGGA |
| | | | | | TGCCATGAACCG |
| | | | | | AGACTGCGGAAT |
| | | | | | TCCACTCAGTCA |
| | | | | | TTTGCAGGTAGA |
| | | | | | TGGAGGAATGAC |
| | | | | | CAACAACAAAATT |
| | | | | | CTTATGCAACTA |
| | | | | | CAAGCAGACATT |
| | | | | | CTATATATCCCA |
| | | | | | GTAGTGAAGCCC |
| | | | | | TCGATGCCAGAA |
| | | | | | ACAACTGCCCTG |
| | | | | | GGAGCTGCCATG |
| | | | | | GCAGCCGGGGC |
| | | | | | TGCGGAGGGAGT |
| | | | | | TGGTGTTTGGAG |
| | | | | | TCTTGAACCCGA |
| | | | | | GGATCTGTCAGC |
| | | | | | AGTCACGATGGA |
| | | | | | GCGATTTGAACC |
| | | | | | CCAGATCAATGC |
| | | | | | TGAGGAAAGTGA |
| | | | | | AATTCGTTACTCT |
| | | | | | ACATGGAAGAAG |
| | | | | | GCTGTGATGAAG |
| | | | | | TCAGTGGGCTGG |
| | | | | | GTTACAACTCA |
| Transketolase | CfaAffx.13684.1. | <0.01 | Homo sapiens | 86.53846 | GAAGATCTGGCC |
| | S1_s_at | | transketolase | | ATGTTTCGGTCC |
| | | | (Wernicke-Korsakoff | | ATCCCCACTGCT |
| | | | syndrome); mRNA | | ACGATCTTTTACC |
| | | | (cDNA clone | | CAAGTGACGGGG |
| | | | MGC: 15349 | | TGTCAACAGAGA |
| | | | IMAGE: 4310396); | | AGGCGGTGGAAT |
| | | | complete cds | | TAGCAGCCAATA |
| | | | | | CAAAGGGCATCT |
| | | | | | GCTTCATCCGGA |
| | | | | | CCAGCCGCCCAG |
| | | | | | AAAACGCCATCA |
| | | | | | TCTATAACAACAA |
| | | | | | TGAGGATTTCCA |
| | | | | | AATCAAACAAGC |
| | | | | | CAAGGTGGTCCT |
| | | | | | GAAGAGCAAGGA |
| | | | | | TGACCAGGTGAC |
| | | | | | TGTGATTGGGGC |
| | | | | | CGGAGTGACCCT |
| | | | | | ACATGAGGCCTT |
| | | | | | GGCTGCTGCTGA |
| | | | | | ACTGCTGAAGAA |
| | | | | | AGAGAAGATCAA |
| | | | | | CATTCGTGTGTT |
| | | | | | GGACCCCTTCAC |
| | | | | | CATCAAGCCCCT |
| | | | | | GGACAGAAATCT |
| | | | | | CATTCTCGAAAG |
| | | | | | CGCCCGTGCGAC |
| | | | | | CAAGGGCAGGAT |
| | | | | | CGTCACCGTGGA |
| | | | | | GGACCATTACTA |
| | | | | | TGAAGGTGGCAT |
| | | | | | AGGTGAGGCAGT |
| | | | | | GTCCTCTGCCTT |
| | | | | | GGTGGGTGAGC |
| | | | | | CTGGCATCACCG |
| | | | | | TCTCCCGCCTTG |
| | | | | | CAGTTGGTGAGG |
| | | | | | TACCAAGAAGCG |
| | | | | | GGAAGCCAGCTG |
| | | | | | AGCTGCTGAAGA |
| | | | | | TGTTTGGCATTG |
| | | | | | ACAGGGACGCCA |
| | | | | | TCGCACAAGCTG |
| | | | | | TGAGGGACCTTG |
| | | | | | TCGCCAA |
| Ribulose | Cfa.13084.1.A1_s_at | <0.01 | Homo sapiens SLIT- | 57.79468 | CCCCAAGGAGAT |
| phosphate 3- | | | ROBO Rho GTPase | | GAGGAGCGATGA |
| epimerase | | | activating protein 2 | | CCCCAGCAACAG |
| | | | (SRGAP2); mRNA | | GAANAACAGCCC |
| | | | | | ACTGAAGGGCTG |
| | | | | | GTGTGTGTGTNC |
| | | | | | TTCACGTGCCAG |
| | | | | | AAGAGAAGTTTA |
| | | | | | GATCCTCCCAGG |
| | | | | | GGAATCGCAATG |
| | | | | | TTGTGGCGTCCT |
| | | | | | GACTTGTATGTC |
| | | | | | ACGTTTTGTGTAA |
| | | | | | AAATGGTATATTC |
| | | | | | TTTAAAATAGTGT |
| | | | | | TGATAACTGGAA |
| | | | | | TATTGTATGTATG |
| | | | | | CTTGGAGATGCT |
| | | | | | TTGTGTGAACCT |
| | | | | | AAGACTGTCACT |
| | | | | | CAACAGATGTTG |
| | | | | | GATTGGG |
| Ribose 5- | Cfa.335.2.S1_at | <0.01 | PREDICTED: Canis | 100 | AGCCTTTCTACT |
| phosphate | | | familiaris similar to | | GACCCTGCAAGA |
| isomerase | | | ribose 5-phosphate | | GTGGAGCGTGTT |
| | | | isomerase A (ribose | | CACCTTGAACCC |
| | | | 5-phosphate | | CCAGCGTGCAGC |
| | | | epimerase) | | TGAGGTAGACAT |
| | | | (LOC475755); partial | | GCCTCTCCAGGA |
| | | | mRNA | | GCCTTTGCCTTA |
| | | | | | ATGCATCTGTGC |
| | | | | | CAGACAGACGGC |
| | | | | | TGG |
| Cytochrome c | CfaAffx.4942.1.S1_s_at | <0.01 | PREDICTED: Canis | 100 | GGCAGTTTGAAA |
| oxidase | | | familiaris similar to | | ATAAAGTTCCAG |
| polypeptide VIIa- | | | cytochrome c | | AGAAACAAAAGC |
| liver/heart, | | | oxidase; subunit 7a 3 | | TATTTCAGGAGG |
| mitochondrial | | | (LOC611134); mRNA | | ATAATGGAATTC |
| precursor | | | | | CAGTGCATCTAA |
| | | | | | AGGGTGGAGTAG |
| | | | | | CTGATGCCCTCC |
| | | | | | TGTATAGAGCCA |
| | | | | | CTATGATGCTTA |
| | | | | | CAGTTGGTGGAA |
| | | | | | CAGCATATGCCA |
| | | | | | TGTATCAGCTAG |
| | | | | | CTGTGGCTTCTT |
| | | | | | TTCCCAAGAAGCA |
| Cytochrome c | Cfa.15065.1.S1_at | <0.01 | PREDICTED: Canis | 99.75961 | GGTCCGCAGTCG |
| oxidase subunit | | | familiaris similar to | | TTCTGTGCGGTC |
| VIII liver form | | | Cytochrome c oxidase | | ATGTCTGTGCTG |
| | | | polypeptide VIII-liver; | | GTGCCGCAGCTG |
| | | | mitochondrial | | CTGAGGGGCCTA |
| | | | precursor | | ACAGGCCTCACC |
| | | | (Cytochrome c | | CGGCGGCTCCC |
| | | | oxidase subunit 8-2) | | GGTGCATCGTGC |
| | | | (LOC476040); mRNA | | CCAGATCCATTC |
| | | | | | CAAGCCGCCGC |
| | | | | | GGGAGCAGCTC |
| | | | | | GGGACCATGGAT |
| | | | | | GTTGCCGTTGGG |
| | | | | | CTCACCTNCTGC |
| | | | | | TTCCTGTGTTTCC |
| | | | | | TCCTGCCATCGG |
| | | | | | GCTGGGTCCTGT |
| | | | | | CACACCTGGAGA |
| | | | | | GCTACAAGAAGC |
| | | | | | GGGAGTGAAGG |
| | | | | | GGGCTGTCCTGT |
| | | | | | CCCTCACCCTGT |
| | | | | | GACCTGACCACC |
| | | | | | CCTGGCCTGTCC |
| | | | | | TGATCATGTCTG |
| | | | | | CTGCATTCCTGG |
| | | | | | CCGGCCTTCCAT |
| | | | | | GGATCATGTCCT |
| | | | | | TCAATTACAGTG |
| | | | | | ACCTCTTCTACA |
| | | | | | GTCATGACCTCT |
| | | | | | TGATTTCTCCATG |
| | | | | | GTGACATCCTGG |
| | | | | | GACCAAACATAT |
| | | | | | TGGTTTATAA |
| Ubiquinolucytochrome c | Cfa.1425.2.A1_at | <0.01 | PREDICTED: Canis | 27.18053 | CTTATGCATTCCT |
| reductase | | | familiaris similar to | | TCCAAAATTGGA |
| | | | Ubiquinol- | | TCATTTAGGTCAA |
| | | | cytochrome-c | | ATTATTTGATGTT |
| | | | reductase complex | | AAATCATAAGTTT |
| | | | core protein 2; | | TCATTTGCTTACA |
| | | | mitochondrial | | TTTACGATATCAG |
| | | | precursor (Complex III | | CGTCAGCTACGG |
| | | | subunit II); transcript | | AATCAATCTGCT |
| | | | variant 1 | | GAAGGACCGTGG |
| | | | (LOC479815); mRNA | | CTGGCGGCGTGT |
| | | | | | ACGATCCAGCAA |
| | | | | | CCAGCGCCTGG |
| | | | | | GACCCGACTTCA |
| | | | | | TCCAGGAACCCC |
| | | | | | TCAGAAGACTCC |
| | | | | | ACTGACATTAGG |
| | | | | | AAGACTCATAAG |
| | | | | | AACCTTACAAGA |
| | | | | | AAAAGTATCAAC |
| | | | | | CCCATCAAAACG |
| | | | | | GCAGAAAAGAAA |
| | | | | | CATATCTTGTTAT |
| | | | | | TAGTAGCTGAAA |
| | | | | | TTCCATTTTCTAC |
| | | | | | ATGTTGCCATAC |
| | | | | | CTTATAAAAACTA |
| | | | | | CACTAAGCTACG |
| | | | | | CTTAAGGAAATA |
| | | | | | CATTTTCTTAAAT |
| | | | | | AAATTAGAATTGA |
| | | | | | AACCAATTTTTAA |
| | | | | | GTAAATCTAGGG |
| | | | | | NTTCAATTTATTC |
| | | | | | TCATTGNGTNTT |
| | | | | | GTTTCTGGTGCA |
| | | | | | ATCATGAANAAC |
| | | | | | AGCATNCTATTAA |
| | | | | | CCAACCTTGGTC |
| | | | | | CCATGTACATAA |
| ATP synthase | CfaAffx.3186.1.S1_s_at | <0.01 | PREDICTED: Canis | 98.57651 | AATTGGGACTGT |
| | | | familiaris similar to | | GTTTGGGAGCCT |
| | | | ATP synthase; H+ | | CATCATTGGTTAT |
| | | | transporting; | | NCCAGGAATCCC |
| | | | mitochondrial F0 | | TCTCTGAAGCAA |
| | | | complex; subunit c | | CAGCTCTTCTCC |
| | | | isoform 2a precursor | | TACGCCATTCTG |
| | | | (LOC477595); mRNA | | GGCTTTGCCCTC |
| | | | | | NCGGAGGCCATG |
| | | | | | GGGCTTTTTTGC |
| | | | | | CTGATNGTGGCC |
| | | | | | TTTCTCATCCTCT |
| | | | | | TNGCCATGTGAA |
| | | | | | GGAGTCGTCTCC |
| | | | | | ACCTCCCATAGG |
| | | | | | TCTTTCTCCCATG |
| | | | | | TCTTGTCTGCCC |
| | | | | | TGTATGCCCTGT |
| | | | | | ATGTTCCTTTTCC |
| | | | | | TATACCTCCCCA |
| | | | | | GGCAGCCTGGG |
| | | | | | GAAAGTGGTTGG |
| | | | | | CTCAGGGTTTGA |
| | | | | | CA |
| NADH- | Cfa.4415.1.S1_at | <0.01 | PREDICTED: Canis | 98.20789 | GGTGACTTTGGA |
| ubiquinone | | | familiaris similar to | | CGTCCGTTCCTG |
| oxidoreductase | | | NADH-ubiquinone | | CTCTGTGGAGGC |
| | | | oxidoreductase | | NNTGCTTCGTTC |
| | | | MLRQ subunit | | CGGGCCTTGCG |
| | | | (Complex I-MLRQ) | | GCAACTCGGTNT |
| | | | (CI-MLRQ) | | TTCCTTCCCCTG |
| | | | (LOC477682); mRNA | | CGCGGGAGACCT |
| | | | | | CTGCCACAACCA |
| | | | | | TGTTACGCCAGA |
| | | | | | TCATCGGTCAGG |
| | | | | | CCAAGAAGCATC |
| | | | | | CGAGCTTGATCC |
| | | | | | CCCTCTTCATATT |
| | | | | | TATTGGGGCAGG |
| | | | | | AGGTACTGGAGC |
| | | | | | AGCGCTGTATGT |
| | | | | | ATTGCGCTTGGC |
| | | | | | ATTGTTCAATCCA |
| | | | | | GATGTTAGTTGG |
| | | | | | GATAGGAAGAAT |
| | | | | | AACCCAGAACCT |
| | | | | | TGGAACAAACTG |
| | | | | | GGTCCCAATGAT |
| | | | | | CAATACAAGTTCT |
| | | | | | ACTCAGTGAATG |
| | | | | | TAGATTACAGCA |
| | | | | | AACTGAAGAAAG |
| | | | | | AAGGTCCAGACT |
| | | | | | TCTAAATGAAATG |
| | | | | | TTTCACTATAAAG |
| | | | | | CTGCTTAGAATG |
| | | | | | AAGGTCTTCCAG |
| | | | | | AAGCCATCCGCA |
| | | | | | CAATTTTCCACTT |
| | | | | | ATCCAGGAAATA |
| | | | | | TTTCCCCTCTAAA |
| | | | | | TGCACGAAATCA |
| | | | | | TGTTGGTGTATT |
| | | | | | GTGTTGGGGTTT |
| | | | | | ACACTNNANNAN |
| | | | | | TAAATATCTGAAA |
| | | | | | CTTGANANGTGT |
| | | | | | CACTATTTAATGC |
| | | | | | TGAAAATTTGCTC |
| | | | | | TGAACTTTA |
| Facilitated | Cfa.1370.1.A1_at | <0.01 | Homo sapiens cDNA | 23.95833 | TTGGAAGGATGG |
| glucose | | | FLJ44038 fis; clone | | ATGCTTGCCCCA |
| transporter/ | | | TESTI4028880; highly | | GGTCATGGACAC |
| Glucose | | | similar to Glucose | | CTCCACAAATCA |
| transporter-like | | | transporter type 3; | | TCTAGTTTCCCA |
| protein III | | | brain | | GTATTTTTATAAA |
| (GLUT3) | | | | | TGGAGATTGGGC |
| | | | | | TCCATGACACTTT |
| | | | | | ACTTGGTCTTCC |
| | | | | | TTCTTACATAGGT |
| | | | | | TTTTTGATTACCC |
| | | | | | TTTCTCTCCTTGG |
| | | | | | TGCTTATATACTT |
| | | | | | AAGACCCTTTAG |
| | | | | | CCAAACCCTTGC |
| | | | | | CAATGACAGTAT |
| | | | | | TTCAGTCACTAG |
| | | | | | TTCTCACTGTTTC |
| | | | | | CTCTGATCATTG |
| | | | | | AGCCTTTGGAAA |
| | | | | | AAAAATCTCACA |
| | | | | | GAGCTTATATGT |
| | | | | | AATGGGGCTTGG |
| | | | | | TTGAACAGATGA |
| | | | | | CTTCCTGTAACT |
| | | | | | GCACCTCTACTT |
| | | | | | TTGGCTTCTCAA |
| | | | | | AAACAGTGGGTT |
| | | | | | GGCAGTAATGCA |
| | | | | | GCGTGGAAGTTT |
| | | | | | TCCCATTTCTCA |
| | | | | | GTGAC |

TABLE 14
Summary of Genes involved in Glucose Metabolism

| | Gene | |
|---|---|---|
| | Expression | |
| | Compared to | |
| Gene | Control | Role |
| Phosphorylase kinase | ↓ | Necessary for activation of |
| | | glycogen synthase which |
| | | stores glucose as glycogen |
| Phosphorylase | ↓ | Necessary for glycogen |
| | | conversion to glucose 1- |
| | | phosphate which feeds into |
| | | glycolysis |
| Glycogen synthase kinase 3 | ↓ | Necessary for activation of |
| | | glycogen synthase which |
| | | stores glucose as glycogen |
| Calmodulin | ↓ | Necessary for activation of |
| | | glycogen synthase which |
| | | stores glucose as glycogen |
| Protein Kinase C | ↓ | Necessary for activation of |
| | | glycogen synthase which |
| | | stores glucose as glycogen |
| Protein Kinase C Binding | ↓ | Necessary for activation of |
| Protein | | glycogen synthase which |
| | | stores glucose as glycogen |
| Hexokinase 3 | ↓ | Necessary for glucose |
| | | conversion to pyruvate to |
| | | enter the TCA cycle |
| Fructose 1,6 | ↓ | Necessary for glucose |
| bisphosphatase | | conversion to pyruvate to |
| | | enter the TCA cycle |
| Glyceraldehyde 3- | ↓ | Necessary for glucose |
| phosphate dehydrogenase | | conversion to pyruvate to |
| | | enter the TCA cycle |
| Glucose 6-phosphate | ↓ | Involved in pentose |
| dehydrogenase | | phosphate pathway |
| Enolase | ↓ | Necessary for glucose |
| | | conversion to pyruvate to |
| | | enter the TCA cycle |
| Lactate dehydrogenase | ↓ | Involved in converting |
| | | private to lactate |
| Citrate lyase | ↓ | Necessary for citrate |
| | | conversion to oxaloacetate |
| | | which feeds acetyl-CoA |
| | | into the fatty acid synthesis |
| | | pathway |
| Glycerol kinase | ↓ | Necessary for changing |
| | | glycerol into DHAP which |
| | | feeds into glycolysis |
| Transketolase | ↓ | Involved in pentose |
| | | phosphate pathway |
| Ribulose phosphate 3- | ↓ | Involved in pentose |
| epimerase | | phosphate pathway |
| Ribose 5-phosphate | ↓ | Involved in pentose |
| isomerase | | phosphate pathway |
| Cytochrome c oxidase | ↓ | Associated with the |
| polypeptide VIIa- | | production of ATP (energy |
| liver/heart, mitochondrial | | source) in the electron |
| precursor | | transport chain which is |
| | | associated with the TCA |
| | | cycle |
| Cytochrome c oxidase | ↓ | Associated with the |
| subunit VIII liver form | | production of ATP (energy |
| | | source) in the electron |
| | | transport chain which is |
| | | associated with the TCA |
| | | cycle |
| Ubiquinol--cytochrome c | ↓ | Associated with the |
| reductase | | production of ATP (energy |
| | | source) in the electron |
| | | transport chain which is |
| | | associated with the TCA |
| | | cycle |
| ATP synthase | ↓ | Associated with the |
| | | production of ATP (energy |
| | | source) in the electron |
| | | transport chain which is |
| | | associated with the TCA |
| | | cycle |
| NADH-ubiquinone | ↓ | Associated with the |
| oxidoreductase | | production of ATP (energy |
| | | source) in the electron |
| | | transport chain which is |
| | | associated with the TCA |
| | | cycle |
| Facilitated glucose | ↓ | Involved in glucose uptake |
| transporter/Glucose | | |
| transporter-like protein-III | | |
| (GLUT3) | | |

### Example 5

### Comparison of Gene Expression Profiles of Genes Associated with the Aging Process: Healthy Adult Dogs Versus Senior Dogs in Comparison to Control Diet Versus Super Senior Diet

A dog's gene expression profile changes as the dog ages from being an adult dog to becoming a geriatric (senior) dog. This is true for genes associated with numerous biological pathways such as, e.g., glucose metabolism, blood clotting and bone and muscle integrity but also with regard to genes that have been associated with the aging process, or senescence, in general. With regard to this class of “aging” associated genes, we have found that, by feeding senior dogs a super senior diet according to the present invention, the gene expression profile of certain of these genes in lymphocytes tends to move towards the profile of an adult dog from that of a geriatric dog. Thus, geriatric dogs fed a super senior diet according to the present invention can have their genetic profile altered to resemble more closely the genetic profile of a healthy adult dog.

The results displayed below in Tables 15-20, show that genes normally altered with the aging process can be regulated through nutritional strategies targeted at common aging changes. Specifically, the results show that, when fed a super senior diet, generally the expression levels of the genes in lymphocytes move in the opposite direction as that of the expression level in a healthy adult animal compared to the expression level in a geriatric animal. That is, when the expression level in a healthy adult animal is high compared to a geriatric animal (i.e., “down regulated” in the geriatric animal), the super senior fed geriatric animals generally also have higher expression level (altered to be “up regulated”) as compared to a geriatric animal fed the control diet. Similarly, when the expression level in a healthy adult animal is low compared to a geriatric animal (“up regulated” in the geriatric animal), the super senior fed geriatric animals generally also have lower expression level (altered to be “down regulated”) as compared to a control diet fed geriatric animal. Thus, expression levels of aging related genes in geriatric dogs may be beneficially altered when the geriatric dog is fed a super senior diet of the present invention and thus the dogs may therefore lead lives of improved quality.

TABLE 15
Aging Genes Associated With Inflammation

| | | Direction of | |
|---|---|---|---|
| | | Expression | |
| | | | Super |
| | | | Senior |
| | | Adult v. | v. Control |
| Annotation | Probe ID. | Geriatric | Diet |
| C1q and tumor | CfaAffx.26423.1.S1_at | up | down |
| necrosis factor | | | |
| related protein 2 | | | |
| TNFRSF1A- | CfaAffx.31209.1.S1_s_at | down | down |
| associated via | | | |
| death domain | | | |
| isoform 1 | | | |
| T-cell surface | CfaAffx.15424.1.S1_at | down | down |
| antigen CD2 | | | |
| precursor | | | |
| Delta- | Cfa.17192.1.S1_s_at | down | down |
| aminolevulinic | | | |
| acid dehydratase | | | |
| Attractin precursor | CfaAffx.10508.1.S1_at | up | up |
| Cytochrome C | Cfa.16058.1.A1_x_at | up | up |
| oxidase subunit III | | | |
| NEDD4-like | Cfa.8453.1.A1_at | up | up |
| ubiquitin-protein | | | |
| ligase 1 | | | |
| Ubiquitin specific | CfaAffx.23104.1.S1_at | down | down |
| peptidase 11 | | | |
| Cartilage | Cfa/15775.1.A1_at | up | up |
| intermediate layer | | | |
| protein | | | |

TABLE 16
Genes Associated With DNA Repair/Cell Survival

| | | Direction of | |
|---|---|---|---|
| | | Expression | |
| | | | Super Senior |
| | | Adult v. | v. Control |
| Annotation | Probe | Geriatric | Diet |
| Gemin 7 | CfaAffx.7805.1.S1_s_at | down | down |
| Iroquis-class | CfaAffx..16317.1.S1_at | up | down |
| homeodomain | | | |
| protein IRX-4 | | | |
| Chromobox | CfaAffx.9308.1.S1_at | down | down |
| homolog 4 | | | |
| Ubiquitin specific | CfaAffx.23104,1,S1_at | down | down |
| peptidase 11 | | | |
| ADP-ribosylation | Cfa.13803.1.S1_s_at | down | down |
| factor-like 10B | | | |
| Eukaryotic | CfaAffx.4294.1.S1_at | up | up |
| translation initiation | | | |
| factor 5B | | | |
| General | CfaAffx.1649.1.S1_s_at | down | down |
| transcription factoR | | | |
| 11 H. polypeptide 4 | | | |
| NEDD4-like | Cfa,8453.1.A1_at | up | up |
| ubiquitin-protein | | | |
| ligase 1 | | | |
| Poly(ADP- | Cfa.5341.1.A1_at | up | down |
| ribose)polymerase | | | |
| family, member 8 | | | |

TABLE 17
Aging Genes Associated With Fat/Cholesterol Metabolism

| | | Direction of | |
|---|---|---|---|
| | | Expression | |
| | | | Super Senior |
| | | Adult v. | v. Control |
| Annotation | Probe | Geriatric | Diet |
| Phophomevanonate | Cfa.1406.1.S1_s_at | down | down |
| kinase | | | |
| 5-AMP-activated | CfaAffx.13848.1.S1_at | down | down |
| protein kinase, | | | |
| gamma-1 subunit | | | |
| Apoliprotein A-II | Cfa.8770.1.S1_at | down | down |
| precursor | | | |
| C1q and tumor | CfaAffx.26423.1.S1_at | up | down |
| necrosis factor | | | |
| related protein 2 | | | |
| Attractin precursor | CfaAffx.10508.1.S1_at | up | up |

TABLE 18
Aging Genes Associated With Protein Synthesis

| | | Direction of | |
|---|---|---|---|
| | | Expression | |
| | | | Super Senior |
| | | Adult v. | v. Control |
| Annotation | Probe | Geriatric | Diet |
| Branched chain | Cfa.17186.1_s_at | down | down |
| keto acid | | | |
| dehydrogenase E1, | | | |
| alpha polypeptide | | | |
| Seryl-tRNA | CfaAffx.9380.1.S1_s_at | down | down |
| synthesis | | | |
| Mitochondrial 28S | CfaAffx.200.1.S1_at | down | down |
| ribosomal protein | | | |
| S33 | | | |
| Ribosomal protein | CfaAffx.416.1.S1_x_at | down | up |
| S3a | | | |
| 60S ribosomal | CfaAffx.802.1.S1_at | down | up |
| protein L21 | | | |

TABLE 19
Aging Genes Associated With Cell Growth/Death

| | | Direction of | |
|---|---|---|---|
| | | Expression | |
| | | | Super |
| | | Adult v. | Senior v. |
| Annotation | Probe | Geriatric | Control Diet |
| Sorting nexin-9 | Cfa.1874.1.S1_at | up | up |
| Cell growth | Cfa.3200.1.S1_s_at | down | down |
| regulator with | | | |
| RING finger | | | |
| domain 1 | | | |
| Solute carrier | CfaAffx.14864.1.S1_at | up | up |
| family 39 (zinc | | | |
| transporter) | | | |
| Choline kinase | Cfa.8353.1.A1_at | down | down |
| alpha isoform a | | | |
| Kv Channel | Cfa.3460.1.S1_at | up | down |
| interacting protein 2 | | | |
| Ribosomal | CfaAffx.416.1.S1_x_at | down | up |
| protein S3a | | | |
| Ubiquin specific | CfaAffx.23104.1.S1_at | down | down |
| peptidase 11 | | | |

TABLE 20
Genes Altered With Age and Super Senior Diet with
Unknown Functions

| | | Direction of | |
|---|---|---|---|
| | | Expression | |
| | | | Super Senior |
| | | Adult v. | v. Control |
| Annotation | Probe | Geriatric | Diet |
| Protein | Cfa.13958.1.A1_at | up | up |
| KIAA0406 | | | |
| Hypothetical | CfaAffx.12332.1.S1_at | up | up |
| LOC477905 | | | |
| Nitilase 1 | CfaAffx.19716.1.S1_s_at | down | down |
| CG5645-PA | CfaAffx.27184.1.S1_at | down | down |
| Transcribed locus | Cfa.12738.1.A1_at | up | up |
| Transcribed locus | Cfa.18839.1.S1_at | up | down |

What is claimed is:

1. A method for modulating biological functions associated with the aging process of a senior or super senior companion animal comprising feeding the animal a composition comprising:
at least about 9% by weight protein;
at least about 5% by weight fat; and
at least about 0.05% by weight of at least one omega-3 polyunsaturated fatty acid.

2. The method of claim 1, wherein the biological functions associated with the aging process comprises inflammation, DNA repair or cell survival, fat or cholesterol metabolism, protein synthesis, cell growth and cell death.

3. The method of claim 1 wherein the animal is chosen from a cat, a dog, and a horse.

4. A method for modulating biological functions associated with the aging process of a senior or super senior animal comprising feeding the animal a composition comprising:
at least one omega-3 polyunsaturated fatty acid chosen from docosahexaenoic acid and eicosapentaenoic acid;
at least one antioxidant; and
at least one nutrient chosen from choline, manganese, methionine, cysteine, L-carnitine, lysine, and mixtures thereof.

5. The method of claim 4 wherein the omega-3 polyunsaturated fatty acid in the composition is DHA and wherein the composition comprises at least about 0.02% by weight DHA as measured on a dry matter basis.

6. The method of claim 4 wherein the omega-3 polyunsaturated fatty acid in the composition is DHA and wherein the composition comprises about 0.02% to about 0.40% by weight DHA as measured on a dry matter basis.

7. The method of claim 4 wherein the omega-3 polyunsaturated fatty acid in the composition comprises EPA and wherein the composition comprises at least about 0.1% by weight EPA as measured on a dry matter basis.

8. The method of claim 4 wherein the omega-3 polyunsaturated fatty acid in the composition comprises EPA, and wherein the composition comprises about 0.1% by weight to about 1% by weight EPA as measured on a dry matter basis.

9. The method of claim 4 wherein the omega-3 polyunsaturated fatty acid in the composition comprises a mixture of DHA and EPA, and wherein the composition comprises at least about 0.02% by weight DHA and at least about 0.1% by weight EPA on a dry matter basis.

10. The method of claim 4 wherein the composition comprises one or more antioxidants chosen from vitamin E, vitamin C, taurine, beta-carotene, carnitine, lipoic acid, and cystine.

11. The method of claim 4 wherein the composition comprises at least about 500 IU/kg vitamin E, at least about 50 ppm vitamin C and at least about 600 ppm taurine.

12. The method of claim 4 wherein the composition further comprises at least about 1000 ppm choline.

13. The method of claim 4 wherein the composition fed to the animal is an animal treat or an animal toy.

14. The method of claim 4 wherein the composition fed to the animal as a nutritional supplement.

15. A method for modulating biological functions associated with the aging process of a senior or super senior small or regular breed canine comprising feeding the animal a composition comprising:
about 60% to about 70% by weight carbohydrate;
about 15% to about 25% by weight protein chosen from animal protein and vegetable protein;
about 5% to about 7% by weight fat chosen from animal fat and vegetable fat;
about 2.5% to about 4% by weight of at least one omega-3 polyunsaturated fatty acids;
about 1% to about 2% by weight fiber;
about 1% to about 2% by weight minerals; and
about 0.5 to about 1.5% by weight vitamins.

16. A method for modulating biological functions associated with the aging process of a senior or super senior large breed dog, wherein the method comprises feeding the animal a composition comprising: about 60% to about 70% by weight carbohydrate;
about 15% to about 25% by weight protein chosen from animal protein and vegetable protein;
about 5% to about 7% by weight fat chosen from animal fat and vegetable fat;
about 3% to about 5% by weight of at least one omega-3 polyunsaturated fatty acids;
about 1% to about 1.5% by weight fiber;
about 0.5% to about 1% by weight minerals; and
about 0.75 to about 1.25% by weight vitamins.

17. A method for modulating biological functions associated with the aging process of a senior or super senior cat, wherein the method comprises feeding the animal a composition comprising:
about 30% to about 35% by weight carbohydrate;
about 40% to about 50% by weight protein chosen from animal protein and vegetable protein;
about 12% to about 15% by weight fat chosen from animal fat and vegetable fat;
about 1% to about 2% by weight of at least one omega-3 polyunsaturated fatty acids;
about 3% to about 5% by weight fiber;
about 1% to about 2% by weight minerals; and
about 1% to about 2% by weight vitamins.

18. The method of claim 1 wherein the method comprises feeding the animal the composition in an amount effective to modulating biological functions associated with the aging process, wherein modulation of biological functions associated with the aging process is evidenced by improvement in one or more biological pathways chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport.

19. The method of claim 1 wherein the method comprises feeding the animal the composition in an amount effective to modulating biological functions associated with the aging process, wherein modulation of biological functions associated with the aging process is evidenced by a beneficial change in expression of one or more genes which encode proteins associated with or related to biological pathways chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport.

20. A method to treat an animal suffering from a disorder or disease associated with or related to aging chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport comprising administering to said animal an effective amount of a composition comprising at least about 9% by weight protein, at least about 5% by weight fat, and at least about 0.05% by weight of at least one omega-3 polyunsaturated fatty acid.

21. The method of claim 20 wherein said composition further comprises at least one omega-3 polyunsaturated fatty acid chosen from docosahexaenoic acid (“DHA”) and eicosapentaenoic acid (“EPA”).

22. The method of claim 20 wherein said composition further comprises at least one antioxidant and at least one nutrient chosen from choline, manganese, methionine, cysteine, L-carnitine, lysine, and mixtures thereof.

23. A method to treat an animal suffering from a disorder or disease associated with or related to a biological pathway chosen from blood clotting and platelet activation and aggregation, bone and muscle integrity, inflammatory responses, cartilage degradation and pain response, DNA damage and repair pathways, neural function, glycogen synthesis and degradation, glycolysis, gluconeogenesis, the pentose phosphate pathway, the aging process, and electron transport comprising administering to said animal an effective amount of a composition comprising the components disclosed in Table 1 or Table 1A.

24. The method of claim 1 wherein the method further comprises measuring the enhancement in the quality of life of said animal comprising quantitating the gene expression levels of one or more genes chosen from those disclosed in Tables 5-14 in said animal prior to and after feeding said composition and comparing said levels in the animal wherein an enhancement in the quality of life of said animal is reflected by a beneficial change in gene expression levels in said animal.

25. The method of claim 1 wherein the method comprises feeding the animal the composition in an amount effective to enhance the animal's quality of life, wherein enhanced quality of life is evidenced by a beneficial change in expression of one or more aging genes which encode proteins associated with or related to biological pathways chosen from inflammation, DNA repair, cell survival, fat or cholesterol metabolism, protein synthesis, cell growth and cell death.

26. The method of claim 31 wherein the change in expression is in one or more genes listed on Tables 15-19 and wherein the change in expression is towards the expression level in a healthy adult companion animal as compared to the expression level in a geriatric animal.

27. The method of claim 32 wherein said animal is a dog.

28. The method of claim 1 wherein the method comprises feeding the animal the composition in an amount effective to enhance the animal's quality of life, wherein enhanced quality of life is evidenced by a change in expression of one or more genes listed on Table 20 and wherein the change in expression is towards the expression level in a healthy adult animal as compared to the expression level in a geriatric animal.

29. The method of claim 34 wherein said animal is a dog.

30. A methods of altering the expression of at least one peptide in a mammal, the method comprising administering to the mammal a composition comprising:
at least about 9% by weight protein;
at least about 5% by weight fat; and
at least about 0.05% by weight of at least one omega-3 polyunsaturated fatty acid,
wherein the at least one peptide is selected from the group consisting of X, Y and Z.

31. The method of claim 30, wherein the mammal is a dog, cat or horse.

32. The method of claim 30, wherein the mammal is a geriatric mammal.

33. The method of claim 30, wherein the composition further comprises at least one of an antioxidant, choline, manganese, methionine, cysteine, L-carnitine, lysine or a combination thereof.

34. The method of claim 30, wherein the expression of the at least one gene is increased.

35. The method of claim 36, wherein the expression of the at least one gene is decreased.

36. A method for screening one or more test compounds for its ability to alter the expression of at least one gene of interest in a mammal, the method comprising
a) administering a control composition to a control group of mammals and determining the levels of expression of the at least one gene of interest,
b) administering the one or more test compositions to an experimental group of mammals and determining the levels of expression of the least one gene of interest, wherein the test composition comprises at least about 9% by weight protein; at least about 5% by weight fat; and at least about 0.05% by weight of at least one omega-3 polyunsaturated fatty acid, and
c) determining the differences in expression levels in the at least one gene of interest between the control and experimental groups of mammals after each group has been administered their respective compositions,
wherein a difference in the expression levels of the at least one gene of interest indicates that the test composition is capable of altering the expression of the at least one gene of interest.

37. The method of claim 36, where the difference indicates that levels of expression of the at least one gene of interest is increased in the experimental group compared to the control group.

38. The method of claim 36, where the difference indicates that levels of expression of the at least one gene of interest is decreased in the experimental group compared to the control group.

39. The method of claim 36, wherein the at least one gene of interest is selected from the group consisting of X, Y and Z.

40. The method of claim 36, wherein the levels of expression of more than one gene of interest are determined.

41. A method for screening one or more test compounds for its ability to alter the expression of at least one gene of interest in a mammal, the method comprising
a) administering a control composition to a control group of mammals and determining the levels of expression of the at least one gene of interest, wherein the control composition comprises at least about 9% by weight protein; at least about 5% by weight fat; and at least about 0.05% by weight of at least one omega-3 polyunsaturated fatty acid,
b) administering the one or more test compositions to an experimental group of mammals and determining the levels of expression of the least one gene of interest, and
c) determining the differences in expression levels in the at least one gene of interest between the control and experimental groups of mammals after each group has been administered their respective compositions,
wherein a difference in the expression levels of the at least one gene of interest indicates that the test composition is capable of altering the expression of the at least one gene of interest.

42. The method of claim 41, where the difference indicates that levels of expression of the at least one gene of interest is increased in the experimental group compared to the control group.

43. The method of claim 41, where the difference indicates that levels of expression of the at least one gene of interest is decreased in the experimental group compared to the control group.

44. The method of claim 41, wherein the at least one gene of interest is selected from the group consisting of X, Y and Z.

45. The method of claim 44, wherein the levels of expression of more than one gene of interest are determined.
